(19) United States
(12) Patent Application Publication
(10) Pub. No.: US 2009/0209618 A1
(43) Pub. Date: Aug. 20, 2009

(54) Pyruvate dehydrogenase kinases as therapeutic targets for cancer and ischemic diseases

(76) Inventors: Chi V. Dang, Baltimore, MD (US); Jung-Whan Kim, Baltimore, MD (US); Gregg Semenza, Reisterstown, MD (US)

Correspondence Address: EDWARDS ANGELL PALMER & DODGE LLP, P.O. BOX 55874, BOSTON, MA, 02205, US

(21) Appl. No.: 11/664,883

(22) PCT Filed: Oct. 6, 2005
(86) PCT No.: PCT/US05/36067

§ 371 Date: Feb. 9, 2009

Related U.S. Application Data
(60) Provisional application No. 60/617,610, filed on Oct. 8, 2004.
(60) Provisional application No. 60/698,795, filed on Jul. 13, 2005.

(51) Int. Cl. A61K 31/7105; A61K 31/7088; C12N 15/11; C12N 5/06; C12Q 1/68; C12Q 1/02

(52) U.S. Cl. 514/44 A; 435/320.1; 435/325; 435/6; 435/29; 514/557; 514/255.01; 514/686

(57) ABSTRACT

The invention provides therapeutic and prophylactic compounds and methods for altering the activity of pyruvate dehydrogenase kinase (e.g. PDK1, PDK2, PDK3, PDK4). Such therapies are useful for the treatment of neoplasia. The invention further provides therapeutic and prophylactic compounds and methods of altering pyruvate dehydrogenase activity to treat or prevent cell death related to ischemia.

### REFERENCE TO RELATED APPLICATIONS

This application claims benefit of U.S. Provisional Application Ser. No. 60/617,610, filed on Oct. 8, 2004, and U.S. Provisional Application Ser. No. 60/698,795, filed on Jul. 13, 2005, the contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: HV28180 and CA51497. The government may have certain rights in the invention.

### BACKGROUND OF THE INVENTION

Oxygen homeostasis is critically important for the survival of all mammalian cells. In the absence of sufficient oxygen, normal cellular metabolism is impaired. Hypoxia-inducible factor-1 alpha (HIF-1alpha) plays an essential role in cellular and systemic O₂ homeostasis by regulating the expression of a number of genes, including genes that function in glycolysis. HIF-1 alpha is thought to be a component of the cellular response to hypoxia and ischemia under pathophysiological conditions, such as stroke. During stroke an acute interruption or reduction of cerebral blood flow reduces available oxygen to the nervous system and causes either focal or global brain damage, with characteristic biochemical and molecular alterations.

Maintenance of oxygen levels is particularly important during periods of rapid cellular proliferation. During neoplastic cell proliferation, for example, O₂ requirements in the proliferating neoplastic cell mass exceed the available O₂ supply. Hypoxia develops in the majority of solid tumors due to the inability of the existing vasculature to supply the growing tumor mass. Tumor cells use several mechanisms to survive in low oxygen tension. One strategy involves the activation of genes downstream of HIF1. Clinical evidence suggests that intratumoral hypoxia correlates with an increase in the aggressiveness of neoplastic cells and their resistance to existing therapies, leading to poor patient prognoses. Methods of treating such aggressive neoplasias are urgently required.

### SUMMARY OF THE INVENTION

As described below, the invention provides therapeutic and prophylactic compounds and methods for altering the activity of pyruvate dehydrogenase kinase (e.g., PDK1, PDK2, PDK3, PDK4). Such therapies are useful for the treatment of neoplasia. The invention further provides therapeutic and prophylactic compounds and methods of altering pyruvate dehydrogenase to treat or prevent cell death related to hypoxia.

In one aspect, the invention features a method of treating or preventing a neoplasia in a subject (e.g., mammal, such as a human). The method involves administering to a subject in need of such treatment an effective amount of a pharmaceutical composition containing a PDK inhibitor in a pharmaceutically acceptable inhibitor.

In another aspect, the invention features a method of treating or preventing a neoplasia. The method involves administering to a patient in need of such treatment an effective amount of a pharmaceutical composition that decreases the expression of a PDK polypeptide.

In a related aspect, the invention features a method of treating or preventing a neoplasia in a subject. The method involves administering to a subject in need of such treatment an effective amount of a pharmaceutical composition that decreases the biological activity of a PDK polypeptide.

In another related aspect, the invention features a method of treating or preventing a neoplasia in a subject. The method involves administering to a subject in need of such treatment an effective amount of a pharmaceutical composition that decreases the expression of a PDK nucleic acid molecule.

In yet another related aspect, the invention features a method of treating or preventing a neoplasia in a subject. The method involves administering to a subject in need of such treatment an effective amount of a pharmaceutical composition containing a PDK inhibitory nucleic acid molecule formulated in a pharmaceutically acceptable carrier.

In yet another related aspect, the invention features a method of treating or preventing a neoplasia in a subject. The method involves administering to a subject in need of such treatment an effective amount of a pharmaceutical composition containing a PDK1 inhibitor in a pharmaceutically acceptable inhibitor.

In another aspect, the invention features a method of treating or preventing a neoplasia. The method involves administering to a subject in need of such treatment an effective amount of a pharmaceutical composition that decreases the expression of a PDK1 polypeptide.

In a related aspect, the invention features a method of treating or preventing a neoplasia in a subject. The method involves administering to a subject in need of such treatment an effective amount of a pharmaceutical composition that decreases the biological. activity of a PDK1 polypeptide.

In another aspect, the invention features a method of treating or preventing a neoplasia in a subject. The method involves administering to a subject in need of such treatment an effective amount of a pharmaceutical composition that decreases the expression of a PDK1 nucleic acid molecule.

In another aspect, the invention features a method of treating or preventing a neoplasia in a subject. The method involves administering to a subject in need of such treatment an effective amount of a pharmaceutical composition containing a PDK1 inhibitory nucleic acid molecule, formulated in a pharmaceutically acceptable carrier. In one embodiment, the inhibitory nucleic acid molecule is a PDK1 siRNA. In another embodiment, the siRNA has the following sequence: 5′-CUACAUGAGUCGCAUUUCAdTdT-3.′

In another aspect, the invention features a PDK inhibitory nucleic acid molecule containing at least ten nucleic acids complementary to a nucleic acid molecule encoding a PDK polypeptide selected from the group consisting of PDK1, PDK2, PDK3, and PDK4, where the nucleic acid molecule inhibits expression of the PDK polypeptide in the cell. In one embodiment, the molecule contains the nucleotide sequence of a PDK polypeptide selected from the group consisting of PDK1, PDK2, PDK3, and PDK4, or a complement thereof. In another embodiment, the molecule consists essentially of a nucleotide sequence encoding a PDK polypeptide selected from the group consisting of PDK1, PDK2, PDK3, and PDK4, or a fragment thereof, or a complement thereof. In yet another embodiment, the molecule is a double stranded RNA molecule that decreases PDK1, PDK2, PDK3, or PDK4 expression in a cell by at least 10%. In yet another embodiment, the molecule is a siRNA molecule that contains at least 15 nucleic acids of a PDK1, PDK2, PDK3, or PDK4 nucleic acid molecule and decreases expression in the cell by at least 20%. In yet another embodiment, the inhibitory nucleic acid molecule reduces PDK1 expression and contains or consists of the following sequence: 5′-CUACAUGAGUCGCAUUUCAdTdT-3′. In another embodiment, the molecule is an antisense nucleic acid molecule that is complementary to at least six nucleotides of the PDK1 nucleic acid molecule and decreases expression in a cell by at least 10%.

In another aspect, the invention features a vector containing a nucleic acid molecule that encodes a PDK1 inhibitory nucleic acid molecule of any one of claims 26-33. In one embodiment, the vector is a viral vector (e.g., a retroviral, adenoviral, or adeno-associated viral vector). In another embodiment, the PDK inhibitory nucleic acid molecule reduces PDK1 expression and contains the following sequence: 5′-CUACAUGAGUCGCAUUUCAdTdT-3.′

In another aspect, the invention features a vector containing a nucleic acid molecule encoding a PDK polypeptide selected from the group consisting of PDK1, PDK2, PDK3, and PDK4, where the PDK polypeptide is positioned for expression. In another embodiment, the vector is a viral vector (e.g., pMSCVpuro vector). In another embodiment, the vector contains a nucleic acid molecule encoding a PDK polypeptide.

In another aspect, the invention features a host cell (e.g., in vitro or in vivo) containing the vector of any previous aspect. In one embodiment, the cell is a mammalian cell (e.g., a human cell or a murine cell, such as a murine embryonic fibroblast). In another embodiment, the cell is a neoplastic cell (e.g., a P493-6 cell).

In another aspect, the invention features a pharmaceutical composition for the treatment of a neoplasia. In one embodiment, the composition contains a pharmaceutical excipient and an effective amount of a small compound that inhibits a PDK biological activity.

In a related aspect, the invention features a pharmaceutical composition for the treatment of a neoplasia, the composition containing a pharmaceutical excipient and an effective amount of a small compound (e.g., dichloroacetate, 2,2-dichloroacetophenone, and (+)-1-N-[2,5-(S,R)-dimethyl-4-N-(4-cyanobenzoyl)piperazine]-(R) -3,3,3-trifluoro-2-hydroxy-2-methylpropanamide) that inhibits a PDK1 biological activity. In one embodiment, the small compound is and the composition is labeled for the treatment of a neoplasia.

In another aspect, the invention features a pharmaceutical composition for the treatment of a neoplasia containing a pharmaceutical excipient and an effective amount of a PDK nucleic acid inhibitor or portion thereof of any previous aspect.

In a related aspect, the invention features a pharmaceutical composition for the treatment of a neoplasia containing a pharmaceutical excipient and an effective amount of a PDK1 nucleic acid inhibitor or portion thereof of any one of any previous aspect.

In another aspect, the invention features a PDK biomarker purified on a solid substrate, where the PDK biomarker is selected from the group consisting of PDK1, PDK2, PDK3, and PDK4.

In another aspect, the invention features a PDK1 biomarker purified on a solid substrate.

In another aspect, the invention features a diagnostic kit for the diagnosis of a neoplasia in a subject containing a PDK nucleic acid molecule, or fragment thereof, and written instructions for use of the kit for detection of a neoplasia.

In a related aspect, the invention features a diagnostic kit for the diagnosis of a neoplasia in a subject containing an antibody that specifically binds a PDK polypeptide selected from the group consisting of PDK1, PDK2, PDK3, and PDK4, or a fragment thereof, and written instructions for use of the kit for detection of a neoplasia.

In another aspect, the invention features a diagnostic kit for the diagnosis of a neoplasia in a subject containing an antibody that specifically binds a phosphorylated PDH polypeptide, or a fragment thereof, and written instructions for use of the kit for detection of a neoplasia.

In a related aspect, the invention features a diagnostic kit for the diagnosis of a neoplasia in a subject containing an adsorbent, where the adsorbent retains a PDK1, PDK2, PDK3, or PDK4 biomarker, and written instructions for use of the kit for detection of a neoplasia.

In another related aspect, the invention features a diagnostic kit for the diagnosis of a neoplasia in a subject containing an adsorbent, where the adsorbent retains a phosphorylated PDH polypeptide, and written instructions for use of the kit for detection of a neoplasia In yet another related aspect, the invention features a diagnostic kit for the diagnosis of a neoplasia in a subject containing reagents for measuring a PDK1, PDK2, PDK3, or PDK4 biological activity and directions for the use of the kit in diagnosing neoplasia. In one embodiment, the kit measures the conversion of pyruvate to acetyl coA, PDH phosphorylation, or aerobic or anaerobic respiration in a sample.

In another aspect, the invention features a method of determining the severity of a neoplasia in a patient, The method involves determining PDK1, PDK2, PDK3, or PDK4 activity or expression in a patient sample, where an increase in the level of PDK1, PDK2, PDK3, or PDK4 activity or expression relative to the level of activity or expression in a reference indicates the severity of neoplasia in the patient.

In another aspect, the invention features a method of determining the severity of a neoplasia in a patient, The method involves determining PDK1 activity or expression in a patient sample, where an increase in the level of PDK1 activity or expression relative to the level of activity or expression in a reference indicates the severity of neoplasia in the patient.

In another aspect, the invention features a method of determining the severity of a neoplasia in a patient, The method involves determining phosphorylated PDH in a patient sample, where an increase in phosphorylated PDH relative to the level in a reference indicates the severity of neoplasia in the patient. In one embodiment, an increased severity of neoplasia indicates an aggressive treatment regimen.

In another aspect, the invention features a method of monitoring a patient having a neoplasia. The method involves determining the PDK1 activity in a patient sample, where an alteration in the level of PDK1 activity or expression relative to the level of activity or expression in a reference indicates the severity of neoplasia in the patient.

In related embodiments of any of the above aspects, the patient is being treated for a neoplasia.

In another aspect, the invention features a method of identifying a candidate compound that ameliorates a neoplasia. The method involves contacting a neoplastic cell that expresses a PDK polypeptide under hypoxic conditions with a candidate compound, and comparing the level of expression of the polypeptide in the cell contacted by the candidate compound with the level of polypeptide expression in a control cell not contacted by the candidate compound, where a decrease in the expression of the PDK polypeptide identifies the candidate compound as a candidate compound that ameliorates a neoplasia. In one embodiment, the PDK polypeptide is selected from the group consisting of PDK1, PDK2, PDK3, and PDK4.

In another aspect, the invention features a method of identifying a candidate compound that ameliorates a neoplasia. The method involves contacting a neoplastic cell that expresses a PDK1 polypeptide under hypoxic conditions with a candidate compound, and comparing the level of expression of the polypeptide in the cell contacted by the candidate compound with the level of polypeptide expression in a control cell not contacted by the candidate compound, where a decrease in the expression of the PDK1 polypeptide identifies the candidate compound as a candidate compound that ameliorates a neoplasia. In one embodiment, the decrease in expression is assayed using an immunological assay, an enzymatic assay, or a radioimmunoassay.

In another aspect, the invention features a method of identifying a candidate compound that ameliorates a neoplasia. The method involves contacting a neoplastic cell that expresses a PDK polypeptide under hypoxic conditions with a candidate compound, and comparing the biological activity of the PDK polypeptide in the cell contacted by the candidate compound with the level of biological activity in a control cell not contacted by the candidate compound, where a decrease in the biological activity of the PDK polypeptide identifies the candidate compound as a candidate compound that ameliorates a neoplasia.

In another aspect, the invention features a method of identifying a candidate compound that ameliorates a neoplasia. The method involves contacting a neoplastic cell that expresses a PDK1 polypeptide under hypoxic conditions with a candidate compound, and comparing the biological activity of the PDK1 polypeptide in the cell contacted by the candidate compound with the level of biological activity in a control cell not contacted by the candidate compound, where a decrease in the biological activity of the PDK1 polypeptide identifies the candidate compound as a candidate compound that ameliorates a neoplasia.

In various embodiments of any of the above aspects, the biological activity is assayed using an immunological assay, an enzymatic assay, or a radioimmunoassay. In other embodiments of any of the above aspects, biological activity is assayed by measuring PDH phosphorylation, by measuring the conversion of pyruvate to acetyl coA, or by measuring aerobic or anaerobic respiration.

In another aspect, the invention features a method of identifying a candidate compound that ameliorates a neoplasia. The method involves contacting a neoplastic cell that expresses a PDK nucleic acid molecule under hypoxic conditions with a candidate compound, and comparing the level of expression of the nucleic acid molecule in the cell contacted by the candidate compound with the level of expression in a control cell not contacted by the candidate compound, where a decrease in expression of the PDK nucleic acid molecule identifies the candidate compound as a candidate compound that ameliorates a neoplasia.

In a related aspect, the invention features a method of identifying a candidate compound that ameliorates a neoplasia. The method involves contacting a neoplastic cell that expresses a PDK1 nucleic acid molecule under hypoxic conditions with a candidate compound, and comparing the level of expression of the nucleic acid molecule in the cell contacted by the candidate compound with the level of expression in a control cell not contacted by the candidate compound, where a decrease in expression of the PDK1 nucleic acid molecule identifies the candidate compound as a candidate compound that ameliorates a neoplasia In one embodiment, the decrease in expression is a decrease in transcription or a decrease in translation.

In yet another aspect, the invention features a method of identifying a candidate compound that ameliorates a neoplasia. The method involves contacting a PDK polypeptide with a candidate compound; and detecting binding of the candidate compound to a PDK polypeptide, where the binding identifies the candidate compound as a compound that ameliorates a neoplasia.

In still another aspect, the invention features a method of identifying a candidate compound that ameliorates a neoplasia. The method involves contacting a neoplastic cell that expresses a PDK polypeptide under hypoxic conditions with a candidate compound; and detecting a decrease in cell survival in the neoplastic cell relative to a corresponding control cell, where the decrease in cell survival identifies the candidate compound as a compound that ameliorates a neoplasia.

In another aspect, the invention features a method of identifying a candidate compound that ameliorates a neoplasia. The method involves contacting a PDK1 polypeptide with a candidate compound; and detecting binding of the candidate compound to a PDK1 polypeptide, where the binding identifies the candidate compound as a compound that ameliorates a neoplasia.

In a related aspect, the invention features a method of identifying a candidate compound that ameliorates a neoplasia. The method involves contacting a neoplastic cell that expresses a PDK1 polypeptide under hypoxic conditions with a candidate compound; and detecting a decrease in cell survival in the neoplastic cell relative to a corresponding control cell, where the decrease in cell survival identifies the candidate compound as a compound that ameliorates a neoplasia. In one embodiment, the cell is selected from the group consisting of an MCF-7, MCF-7ADR, COLO320, HCT116, Ramos, DW6, and P493-6. In another embodiment, the decrease in cell survival is determined by measuring an increase in apoptosis, a decrease in proliferation, or a decrease in cell viability.

In another related aspect, the invention features a method of identifying a candidate compound that enhances cell survival in ischemia, The method involves contacting a cell expressing a PDH polypeptide under hypoxic conditions with a candidate compound; and detecting a decrease in a PDH biological activity, where the decrease in the PDH biological activity identifies the compound as a candidate compound that enhances survival in a cell at risk of cell death related to hypoxia.

In another related aspect, the invention features a method of identifying a candidate compound that treats or prevents cell death related to ischemia, The method involves contacting a cell expressing a PDH polypeptide under hypoxic conditions with a candidate compound; and detecting an increase in cell survival, where the increase in cell survival identifies the compound as a candidate compound that enhances cell survival in ischemia.

In yet another related aspect, the invention features a method of enhancing cell survival in a subject in need thereof, The method involves administering to a subject in need of such treatment an effective amount of a pharmaceutical composition containing a PDH inhibitor in a pharmaceutically acceptable inhibitor. In one embodiment, the subject has or is susceptible to ischemia, transient ischemic attacks, reperfusion injury, traumatic injury, stroke, and myocardial infarction. In another embodiment, the PDH inhibitor is a small molecule (e.g., fluoropyruvate, bromopyruvate, or 2-oxo-3-butynoic acid). In another embodiment, the PDH inhibitor is a nucleic acid inhibitor of PDH expression. In yet another embodiment, the nucleic acid inhibitor is a small interfering RNA (siRNA, antisense RNA, or other nucleic acid inhibitor of PDH expression. In yet another embodiment, the PDH inhibitor is a nucleic acid molecule that encodes PDK.

In another aspect, the invention features a method of treating or preventing cell damage related to hypoxia in a subject. The method involves administering to a subject in need of such treatment an effective amount of a pharmaceutical composition that decreases the expression of a PDH polypeptide.

In a related aspect, the invention features a method of treating or preventing cell damage related to hypoxia in a subject. The method involves administering to a subject in need of such treatment an effective amount of a pharmaceutical composition that decreases the biological activity of a PDH polypeptide. In one embodiment, the biological activity is PDH E1α subunit phosphorylation. In another embodiment, the method involves administering fluoropyruvate, bromopyruvate, or 2-oxo-3-butynoic acid. In another embodiment, the method contains administering a PDK1 polypeptide or a nucleic acid molecule encoding the PDK1 polypeptide to a cell of the subject.

In another aspect, the invention features a method of treating or preventing ischemia in a subject. The method involves administering to a subject in need of such treatment an effective amount of a pharmaceutical composition that decreases the expression of a PDH nucleic acid molecule. In one embodiment, PDH expression is decreased by the administration of a PDH siRNA.

In another aspect, the invention features a method of reducing cell death in a cell at risk thereof, The method involves administering to a cell an effective amount of a compound that decreases the expression of a PDH nucleic acid molecule.

In a related aspect, the invention features a method of reducing cell death in a cell at risk thereof, The method involves administering to a cell an effective amount of a pharmaceutical composition that decreases the expression of a PDH polypeptide.

In another related aspect, the invention features a method of reducing cell death in a cell at risk thereof, The method involves administering to a cell an effective amount of a pharmaceutical composition that decreases the biological activity of a PDH polypeptide.

In various embodiments, the cell is a neuron or a cardiac myocyte. In other embodiments of the previous aspects, the cell is at risk of cell death associated with ischemia, a transient ischemic attack, reperfusion injury, traumatic injury, stroke, or myocardial infarction.

In another aspect, the invention features a PDH nucleic acid inhibitor containing at least ten nucleic acids complementary to a nucleic acid molecule encoding a PDH polypeptide, where the nucleic acid molecule reduces expression of the PDH polypeptide in a cell.

In one embodiment, the nucleic acid inhibitor contains the nucleotide sequence of PDH or a complement thereof. In another embodiment, the nucleic acid molecule consists essentially of a nucleotide sequence of PDH encoding the PDH polypeptide, a fragment thereof, or a complement thereof. In yet another embodiment, PDH expression is reduced by at least 10%. In yet another embodiment, the nucleic acid inhibitor is an siRNA. In yet another embodiment, the siRNA molecule contains at least 15 nucleic acids of a PDH nucleic acid molecule. In still another embodiment, the nucleic acid molecule is an antisense nucleic acid molecule that is complementary to at least six nucleotides of the PDH nucleic acid molecule and decreases expression in a cell by at least 10%.

In another aspect, the invention features a vector containing a nucleic acid molecule that encodes a PDH inhibitory nucleic acid molecule of any previous aspect. In one embodiment, the vector is a viral vector (e.g., a retroviral, adenoviral, or adeno-associated viral vector).

In another aspect, the invention features a pharmaceutical composition for the treatment or prevention of cell damage related to hypoxia, the composition containing a pharmaceutical excipient and an effective amount of a small compound that inhibits a PDH biological activity. In one embodiment, the small compound is fluoropyruvate, bromopyruvate, or 2-oxo-3-butynoic acid.

In another aspect, the invention features a pharmaceutical composition containing a pharmaceutical excipient and a PDH nucleic acid inhibitor or portion thereof of any previous aspect.

In yet another aspect, the invention features a pharmaceutical composition for the treatment or prevention of cell damage related to hypoxia, the composition containing a pharmaceutical excipient and an effective amount of a vector containing a nucleic acid molecule encoding a PDK polypeptide that inhibits PDH biological activity. In one embodiment, the PDK polypeptide is selected from the group consisting of PDK1, PDK2, PDK3, and PDK4. In another embodiment, the PDK polypeptide is PDK1. In another embodiment, the composition increases PDH E1α subunit phosphorylation.

In another aspect, the invention features a method of identifying a candidate compound that enhances survival in a cell at risk of cell death related to hypoxia, The method involves contacting a cell that expresses a PDH polypeptide under hypoxic conditions with a candidate compound, and comparing the level of expression of the polypeptide in the cell contacted by the candidate compound with the level of polypeptide expression in a control cell not contacted by the candidate compound, where a decrease in the expression of the PDH polypeptide identifies the candidate compound as a candidate compound that ameliorates a neoplasia. In one embodiment, the decrease in expression is assayed using an immunological assay, an enzymatic assay, or a radioimmunoassay.

In another aspect, the invention features a method of identifying a candidate compound that enhances survival in a cell at risk of cell death related to hypoxia, The method involves contacting a cell that expresses a PDH polypeptide under hypoxic conditions with a candidate compound, and comparing the biological activity of the PDH polypeptide in the cell contacted by the candidate compound with the level of biological activity in a control cell not contacted by the candidate compound, where a decrease in the biological activity of the PDH polypeptide identifies the candidate compound as a candidate compound that enhances survival in a cell at risk of cell death related to hypoxia. In one embodiment, the biological activity is assayed using an immunological assay, an enzymatic assay, or a radioimmunoassay. In another embodiment, the biological activity is assayed by detecting an alteration in the phosphorylation state of PDH. In another embodiment, the biological activity is assayed by detecting a decrease in reactive oxygen species production, an increase in glycolysis, an increase in ATP production, or an increase in lactate production.

In another aspect, the invention features a method of identifying a candidate compound that enhances survival in a cell at risk of cell death related to hypoxia, The method involves contacting a cell that expresses a PDH nucleic acid molecule under hypoxic conditions with a candidate compound, and comparing the level of expression of the nucleic acid molecule in the cell contacted by the candidate compound with the level of expression in a control cell not contacted by the candidate compound, where a decrease in expression of the PDH nucleic acid molecule identifies the candidate compound as a candidate compound that enhances survival in a cell at risk of cell death related to hypoxia. In one embodiment, the decrease in expression is a decrease in transcription or a decrease in translation.

In another aspect, the invention features a method of identifying a candidate compound that ameliorates a neoplasia. The method involves contacting a PDH polypeptide with a candidate compound; and detecting binding of the candidate compound to a PDH polypeptide, where the binding identifies the compound as a candidate compound that ameliorates a neoplasia.

In another aspect, the invention features a method of identifying a candidate compound that enhances survival in a cell at risk of cell death related to hypoxia, The method involves contacting a PDH polypeptide with a candidate compound; and detecting a decrease in a PDH biological activity, where the decrease in the PDH biological activity identifies the compound as a candidate compound that enhances survival in a cell at risk of cell death related to hypoxia.

In various embodiments of any previous aspect, the PDK inhibitor is a small molecule including any one or more of dichloroacetate, 2,2-dichloroacetophenone, and (+)-1-N-[2,5-(S,R)-dimethyl-4-N-(4-cyanobenzoyl)piperazine]-(R)-3,3,3-trifluoro-2-hydroxy-2-methylpropanamide. PDK inhibitor is an inhibitory nucleic acid molecule that reduces PDK1 expression. In other embodiments of any previous aspect, the inhibitory nucleic acid molecule is a small interfering RNA (siRNA, antisense RNA, short hairpin RNA (shRNA or another nucleic acid inhibitor of PDK or PDH expression. In preferred embodiments, the inhibitory nucleic acid molecule is an siRNA that inhibits PDK (e.g., PDK1, 2, 3, 4, or PDH expression). In preferred embodiments, the PDK inhibitor is an inhibitory nucleic acid molecule that reduces PDK1 expression, such as an siRNA that includes the following nucleic acid sequence: 5′-CUACAUGAGUCGCAUUUCAdTdT-3.′ In various embodiments of any of the above aspects, the PDK biological activity is kinase activity.

### Definitions

By “PDK polypeptide” is meant a polypeptide having pyruvate dehydrogenase kinase activity and having at least 85% amino acid identity to the amino acid sequence of human PDK1, PDK2, PDK3, or PDK4.

By “PDK biological activity” is meant any function of a pyruvate dehydrogenase kinase, such as enzymatic activity, kinase activity, inhibition of the tricarboxylic acid cycle, the enhancement of cell survival under hypoxic conditions, or inhibition of PDH activity.

By “PDK nucleic acid molecule” is meant a polynucleotide that encodes any one of PDK1, 2, 3, or 4.

By “PDK inhibitor” is meant a compound that reduces the biological activity of PDK1, 2, 3, or 4; or that reduces the expression of an mRNA encoding a PDK polypeptide; or that reduces the expression of a PDK polypeptide. Exemplary PDK inhibitors include dichloroacetate, 2,2-dichloroacetophenone, and (+)-1-N-[2,5-(S,R)-dimethyl-4-N-(4-cyanobenzoyl)piperazine]-(R )-3,3,3-trifluoro-2-hydroxy-2-methylpropanamide. For some applications, it may be advantageous to use a PDK inhibitor that selectively inhibits a particular PDK isoform. In one example, a selective PDK inhibitor is ADZ 7545, which is a selective inbitors of PDK2.

By “PDK1 polypeptide” is meant a polypeptide having substantial identity to the amino acid sequence provided at GenBank Accession No. NP_{—}002601, or an active fragment thereof.

By “PDK1 nucleic acid molecule” is meant a nucleic acid sequence encoding a PDK1 polypeptide. One exemplary nucleic acid sequence is provided at GenBank Accession No. NM_{—}002610.

By “PDK1 biological activity” is meant any function of PDK1, such as enzymatic activity, kinase activity, inhibition of the tricarboxylic acid cycle, the enhancement of cell survival under hypoxic conditions, or the inhibition of PDH activity.

By “PDK1 inhibitor” is meant a compound that reduces the biological activity of PDK1, that reduces the expression of an mRNA encoding a PDK1 polypeptide; or that reduces the expression of a PDK1 polypeptide. Exemplary PDK1 inhibitors include dichloroacetate, 2,2-dichloroacetophenone, and (+)-1-N-[2,5-(S,R)-dimethyl-4-N-(4-cyanobenzoyl)piperazine]-(R) -3,3,3-trifluoro-2-hydroxy-2-methylpropanamide.

By “PDH polypeptide” is meant a protein having substantial amino acid identity to the amino acid sequence provided at GenBank Accession No. AAA31853.

By “PDH nucleic acid molecule” is meant a nucleic acid molecule that encodes a PdH polypeptide.

By “PDH biological activity” is meant an enzymatic activity, such as the conversion of pyruvate to acetyl-coenzyme A, or an activity related to cell death under hypoxic conditions.

By “hypoxic conditions” is meant reduced oxygen levels relative to the level required for the maintenance of normal cell metabolism. For example, a cell cultured under 0.5% O₂ is subject to hypoxia, while a cell cultured at 20% O₂ is cultured under normoxic conditions.

By “anti-sense” is meant a nucleic acid sequence, regardless of length, that is complementary to the coding strand or mRNA of a nucleic acid sequence. The anti-sense nucleic acid may contain a modified backbone, for example, phosphorothioate, phosphorodithioate, or other modified backbones known in the art, or may contain non-natural internucleoside linkages.

By “apoptosis” is meant the process of cell death wherein a dying cell displays a set of well-characterized biochemical hallmarks that include cell membrane blebbing, cell soma shrinkage, chromatin condensation, and DNA laddering. Cells that die by apoptosis include neurons (e.g., during the course of a stroke or ischemic injury), cardiomyocytes (e.g., after myocardial infarction or over the course of congestive heart failure).

By “biomarker” is meant a polypeptide or nucleic acid molecule that can be used as a diagnostic indicator of pathology.

By “double stranded RNA” is meant a complementary pair of sense and antisense RNAs regardless of length.

By “an effective amount” is meant the amount of a compound required to prevent, treat, or ameliorate the symptoms of a disease.

By “host cell” is meant a cell that contains a heterologous nucleic acid molecule.

By “inhibitory nucleic acid molecule” is meant a double-stranded RNA, antisense RNA, or siRNA, or portion thereof that reduces the amount of mRNA or protein encoded by a gene of interest. Preferably, the reduction is by at least 5%, more desirable by at least 10%, 25%, or even 50%, relative to an untreated control. Methods for measuring both mRNA and protein levels are well-known in the art; exemplary methods are described herein. The siRNA may contain a modified backbone, for example, phosphorothioate, phosphorodithioate, or other modified backbones known in the art, or may contain non-natural internucleoside linkages

By “fragment” is meant a portion of a protein or nucleic acid (e.g., 15, 20, 25, 50, 75, or 100 amino acids or nucleotides) that is substantially identical to a reference protein or nucleic acid, and retains at least 50% or 75%, more preferably 80%, 90%, or 95%, or even 99% of the biological activity of the reference.

By “promoter” is meant a polynucleotide sufficient to direct transcription.

By “operably linked” is meant a first polynucleotide positioned adjacent to a second polynucleotide that directs transcription of the second polynucleotide.

By “siRNA” is meant a double stranded RNA that complements a region of an mRNA. Optimally, an siRNA is 21, 22, 23, or 24 nucleotides in length and has a 2 base overhang at its 3′ end.

By “subject” is meant a mammal, such as a human, cat, dog, sheep, cow, goat, pig, horse, rat, or mouse.

“Therapeutic compound” means a substance that has the potential of affecting the function of an organism. A therapeutic compound may decrease, suppress, attenuate, diminish, arrest, or stabilize the development or progression of disease, disorder, or infection in a eukaryotic host organism.

### BRIEF DESCRIPTION OF THE DRAWINGS

FIGS. 1A-1F show induction of PDK1 by hypoxia in a HIF-1-dependent manner. FIG. 1A is an immunoblot showing PDK1 protein expression in P493-6 cells following a twenty-two, twenty-nine, or forty-eight hour incubation under normoxic or hypoxic conditions. β-actin is shown as a loading control. FIG. 1B is an immunoblot showing PDK1 induction in P493-6 cells exposed to 100 μM CoCl₂ under non-hypoxic conditions. Total protein staining is shown as a loading control. FIG. 1C is an immunoblot of PDK1 and hexokinase 2 in wild type and Hif1a^{-/-} murine embryonic fibroblasts (MEF) under hypoxic (0.5% O₂) or normoxic (20% O₂) conditions. β-actin is shown as a loading control. FIG. 1D shows the results of a chromatin immunoprecipitation assay of the human PDK1 gene. Real-time PCR quantification of HIF1α binding to regions 1-4 (amplicons) is indicated as the percentage of total input chromatin DNA. Arrows indicate consensus HIF-1 binding site. FIG. 1E is a graph showing the growth curves of wild type and Hif1a^{-/-} murine embryonic fibroblasts in hypoxic conditions (0.5% O₂). Results are average cell numbers from three independent biological experiments. Error bars represent the standard deviation (S.D.). FIG. 1F includes three panels. The top panels are graphs showing the results of a chromatin immunoprecipitation assay of the human VEGF gene. Sheared chromatin from hypoxic (0.1% O₂) or normoxic (20% O₂) P493-6 cells was precipitated with polyclonal anti-HIF1α antibody or control IgG. Regions 1, 2, and 3 are PCR amplicons measured by real-time PCR. Binding is indicated as the percentage of total input chromatin DNA. The bottom panel is a schematic diagram showing the relative positions of amplicons 1, 2, and 3. The relative positions of consensus HIF1 binding sites are indicated using arrows.

FIGS. 2A-2G show the effect of PDK1 on hypoxic responses of Hif1a^{-/-} murine embryonic fibroblasts. FIG. 2A shows three immunoblots of PDK1, HK2, and β-actin protein expression in Hif1a^{-/-} murine embryonic fibroblasts ectopically expressing PDK1 by pMSCVpuro-PDK1 retroviral transduction after twenty-four-seventy-two hours of hypoxia (0.5% O₂). Two independently transduced cell pools with pMSCVpuro-PDK1 retrovirus (#1 and #2) were used. Hif1a^{-/-} murine embryonic fibroblasts and those transduced with empty pMSCVpuro vector were used as controls. β-actin is shown as a loading control. FIG. 2B is a graph showing growth curves of retrovirally transduced Hif1a^{-/-} murine embryonic fibroblasts under hypoxia (0.5% O₂). Results are average cell numbers from four independent biological experiments. Error bars represent the standard deviation. FIG. 2C includes six panels showing the mean percentages of apoptotic cells (Annexin V positive, 7-AAD negative, right lower panel) from three independent experiments (±S.D.) on the indicated cell types. FIGS. 2D and 2E are immunoblots showing the phosphorylation of PDH E1α subunit by PDK1 expression. FIG. 2D shows an analysis of PDH E1α subunit (41 kDa) after two dimensional gel electrophoresis of lysates from the Hif1α^{-/-} murine embryonic fibroblasts (MEF) expressing PDK1 or those transduced with empty vector. Filters were stripped and re-probed for β-actin, which is shown as an inter-gel reference point for the immunoblot alignment. The very far left lane of each panel represents one-dimensional electrophoresis of the lysates. FIG. 2E shows an analysis of phosphorylation of PDHE1α in hypoxic (0.5% O₂) wild-type murine embryonic fibroblasts compared to normoxic (20% O₂) cells. Arrows indicate a phosphorylated form of PDH E1α subunit. pI=isoelectric points. FIGS. 2F and 2G show the forced expression of murine glucose phosphate isomerase (mGPI) does not rescue hypoxic Hif1α^{-/-} murine embryonic fibroblasts. FIG. 2F is a growth curve of the Hif1α−/− murine embryonic fibroblasts (MEFs) overexpressing mGPI under hypoxic (0.5% O₂) or normoxic (20% O₂) conditions. Hif1α^{-/-} murine embryonic fibroblasts transduced with empty vector were used as controls. Cell numbers (mean+S.D.) from two independent experiments, each measured in duplicate are shown. FIG. 2G is a graph showing mGPI mRNA levels measured by real-time RT-PCR using a TaqMan probe.

FIGS. 3A-E show the effect of PDK1 on hypoxia-induced reactive oxygen species production. FIG. 3A is a graph showing intracellular hydrogen peroxide level in wild-type and Hif1a^{-/-} murine embryonic fibroblasts (after seventy-two hours of hypoxia (0.5% O₂) or normoxia (20% O₂). The data are expressed as the mean fluorescence levels from two independent experiments normalized by protein concentration, and shown as normalized (to Hif1a^{-/-} murine embryonic fibroblast) values. Error bars represents standard error of the mean (S.E.M.). FIG. 3B is a graph showing intracellular hydrogen peroxide level in Hif1a^{-/-} murine embryonic fibroblasts ectopically expressing PDK1 or transduced with empty vector after seventy-two hours of hypoxia (0.5% O₂). Four independent experiments were performed and error bars represent S.E.M. FIG. 3C includes eight panels showing DCF (2′,7′-dichlorofluorescein) fluorescence staining of hypoxic Hif1a^{-/-} murine embryonic fibroblasts transduced with indicated retroviruses. Images were captured with identical photographic exposure times from three randomly selected fields. As a positive control, Hif1a murine embryonic fibroblasts ectopically expressing PDK1 were incubated with 200 μM hydrogen peroxide for four hours in hypoxia before staining (right panels). FIG. 3D is a graph showing the growth curves of hypoxic (0.5% O₂, left panel) or normoxic (20% O₂, right panel) Hif1a^{-/-} murine embryonic fibroblasts incubated with 0.1 μM rotenone. Cell numbers (mean+/−S.D.) from two independent experiments, each measured in duplicate are shown. FIG. 3E is a bar graph showing intracellular ATP levels of wild-type murine embryonic fibroblasts, Hif1a^{-/-} murine embryonic fibroblasts ectopically expressing PDK1 or transduced with empty vector after seventy-two hours of hypoxia (0.5% O₂) or normoxia (20% O₂). Values are normalized to those of normoxic Hif1a^{-/-} murine embryonic fibroblasts.

FIGS. 4A-C show the effect of PDK1 reduction on cell proliferation in response to hypoxia. FIG. 4A shows two immunoblots of PDK1 expression in P493-6 cells after electroporation with PDK1 siRNA or control scrambled siRNA in hypoxic (0.1% O₂) or non-hypoxic (20% O₂) conditions. β-actin is shown as a loading control. FIGS. 4B and 4C are graphs showing the growth curves of P493-6 cells electroporated with PDK1 siRNA or control scrambled siRNA in hypoxia (4B) and normoxia (4C). Results are average cell numbers from two independent biological experiments, each measured in duplicate. Error bars represents the S.D.

FIG. 5 is a schematic diagram showing a model of HIF-1 activation of glycolysis and attenuation of glucose respiration through activation of pyruvate dehydrogenase kinase (PDK). Decreased respiration is essential to diminish reactive species (ROS) production from ineffective electron transport under hypoxia.

FIG. 6 shows the effect of dichloroacetate (DCA) on growth of P493-6 cells in hypoxia (0.1% O₂).

FIG. 7 shows lactate accumulation in the media (top panel) or intracellular lactate levels (lower panel) were measured using 2300 STAT plus glucose/lactate analyzer (YSI Life Sciences). Lactate concentrations were normalized to cell number (for lactate accumulated in media) or protein concentration (for intracellular lactate). HIF1α−/− MEFs overexpressing myrAKT were used as a positive control since AKT has been known to induce glycolysis.

FIGS. 8A-8C provide sequences useful in the practice of the invention. FIG. 8A provides the amino acid sequence of human PDK1 (pyruvate dehydrogenase kinase, isoenzyme 1 (GenBank Accession No. NP_{—}002601). FIG. 8B provides the amino acid sequence of PDK2. FIG. 8C provides the amino acid sequence of PDK3. FIG. 8D provides the amino acid sequence of PDK4. FIG. 8E provides a schematic diagram of the pMSCVpuro vector. FIG. 8F provides the nucleic acid sequence of the Clontech pMSCVpuro vector, respectively.

### DETAILED DESCRIPTION OF THE INVENTION

The present invention generally features compositions and methods of altering pyruvate dehydrogenase kinase (e.g., PDK1, PDK2, PDK3, PDK4) activity for the treatment or prevention of neoplasia. In addition, the present invention provides prophylactic and therapeutic methods of altering pyruvate dehydrogenase activity to enhance the survival of cells at risk of cell death related to hypoxia.

As reported in more detail below, pyruvate dehydrogenase kinase was identified as a gene that is highly induced by hypoxia in human neoplastic cells. PDK1 is involved in the regulation of glucose metabolism by the tricarboxylic acid cycle (TCA). Prior to the present discovery, the suppression of the TCA cycle was not thought to be important for cellular adaptation to hypoxia Inhibition of PDK1 induced cell death in a model of Burkitt's lymphoma. While the examples below are directed to PDK1 specifically, one skilled in the art understands that all PDK isoforms share significant structural (i.e, 66-74% amino acid identity) similarities; in addition, all PDK isoforms share a common biological activity (i.e., all isoforms phosphorylate PDH). Given these structural and functional similarities, any PDK isoform can be substituted for PDK1 in the methods of the invention. In addition, compounds that inhibit a PDK isoform PDK1, 2, 3, or 4) are generally useful for the treatment of neoplasia, and are particularly useful for those aggressive neoplasias that have acquired resistance to hypoxia.

PDK1 phosphorylates and inactivates pyruvate dehydrogenase (PDH). Overexpression of PDK1 protected murine embryonic fibroblasts from death induced by hypoxia. Given this observation, it is reasonable to conclude that compounds that reduce PDH activity, as well as compounds or methods that increase PDK1 activity enhance the survival of cells at risk of hypoxic cell death.

### Pyruvate Dehydrogenase Kinase Inhibitors

Pyruvate dehydrogenase kinase inhibitors are known in the art and are described, for example, by Mann et al., Biochimica et Biophysica Acta 1480:283-292, 2000. Pyruvate dehydrogenase kinase catalytic activity is assayed by measuring NADH formation by the pyruvate dehydrogenase multienzyme complex (PDC) (Mann et al., supra), phosphorylation of a tetradecapeptide substrate (Mann et al., supra), by measuring PDK autophosphorylation (Mann et al., supra), by measuring lactate conversion to CO₂ in cultured fibroblasts (Aicher et al., J. Med. Chem. 43:236-249, 2000), by measuring lactate production in fasting animals (Aicher et al., supra), by measuring PDH phosphorylation (as described in Example 1), by measuring PDH activity (Aicher et al. J. Med. Chem. 43:236-249, 2000), or by any other methods known in the art. The PDH activity assay is the most commonly used method for measuring PDK activity.

Known pyruvate dehydrogenase kinase inhibitors include dichloroacetate, halogenated acetophones (e.g., dichloroacetophenone) (Mann et al., supra), adenosine 5′[β,γ-imido] triphosphate (Mann et al., supra), substituted triterpenes (Mann et al., supra), lactones (Mann et al., supra), monochloroacetate (Whitehouse et al., Biochem J 141: 761-774, 1974), dichloroacetate (Whitehouse et al., supra), trichloroacetate (Whitehouse et al., supra), difluoroacetate (Whitehouse et al., supra), 2-chloropropionate (Whitehouse et al., supra), 2,2′-dichloropropionate (Whitehouse et al., supra), 3-chloropropionate (Whitehouse et al., supra), and 3,3,3-trifluoro-2-hydroxy-2-methylpropionamide (Mann et al., supra), SDZ048-619 (Novartis), SDZ060-011 (Novartis), and SDZ225-066 (Novartis, Aicher et al., supra). One preferred PDK1 inhibitor is (+)-1-N-[2,5-(S,R)-dimethyl-4-N-(4-cyanobenzoyl)piperazine]-(R)-3,3,3-trifluoro-2-hydroxy-2-methylpropanamide (Aicher et al., supra). Other preferred PDK inhibitors are dichloroacetate and 2,2-dichloroacetophenone.

### Pyruvate Dehydrogenase Inhibitors

Pyruvate dehydrogenase (PDH) inhibitors, such as fluoropyruvate, bromopyruvate, and 2-oxo-3-butynoic acid, are known in the art. Methods for assaying PDH activity are described, for example, by Aicher et al., J. Med. Chem. 43:236-249, 2000).

### Neoplastic Disease Therapy

Methods of this invention are particularly suitable for administration to humans with neoplastic diseases. The methods comprise administering an amount of a pharmaceutical composition containing a PDK inhibitor in an amount effective to decrease a biological activity of PDK, such as the phosphorylation of PDH, to achieve a desired effect, be it palliation of an existing tumor mass or prevention of recurrence. A tumor comprises one or more neoplastic cells, or a mass of neoplastic cells, and can also encompass cells that support the growth and/or propagation of a cancer cell, such as vasculature and/or stroma. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases. The present invention includes compositions and methods for reducing the growth and/or proliferation of a neoplastic cell, particularly a neoplastic cell resistant to hypoxia, in a subject.

### Methods of Assaying Neoplastic Cell Growth or Proliferation

As reported herein, induction of PDK1 promotes the survival of hypoxic neoplastic cells. Inhibition of PDK1 was found to reduce the survival of neoplastic cells. Accordingly, the invention provides for the identification and use of therapeutic compounds (e.g., dichloroacetate, 2,2-dichloroacetophenone, (+)-1-N-[2,5-(S,R)-dimethyl-4-N-(4-cyanobenzoyl)piperazine]-(R)-3,3,3-trifluoro-2-hydroxy-2-methylpropanamide) that inhibit PDK1 activity for the treatment of neoplasia. Compounds that inhibit PDK are known in the art and are described, for example, by Mann et al., Biochimica et Biophysica Acta 1480:283-292, 2000. Compounds that inhibit PDK are tested for efficacy in inhibiting neoplastic cell growth, preferably under hypoxic conditions. In one approach, a candidate compound is added to the culture media of a neoplastic cell. Cell survival is then evaluated under normoxic and/or hypoxic conditions in the presence or the absence of the compound. A compound that reduces the survival of a cell, particularly under hypoxic conditions, is identified as useful in the methods of the invention. Compounds that selectively reduce the survival of a cell under hypoxic conditions without substantially effecting the survival of a cell under normoxic conditions are particularly useful. Neoplastic cells suitable for such screens include, but are not limited to, MCF-7, MCF-7ADR (van der Horst et al., Int J Cancer. 2005 Jul 1;115(4):519-27), COLO320, HCT116, Ramos, DW6, and P493-6 (Mezquita et al., Oncogene. 24(5):889-901, 2005) cell lines. MCF-7, COLO320, HCT116 and Ramos are available through the ATCC. The selectivity of such compounds suggests that they are unlikely to adversely effect normal cells; thus, such compounds are unlikely to cause the adverse side-effects typically associated with conventional chemotherapeutics. Therapeutics useful in the methods of the invention include, but are not limited to, those that alter a PDK1 biological activity associated with cell proliferation or adaptation to hypoxia or those that have an anti-neoplastic activity.

Selected compounds desirably reduce the survival, growth, or proliferation of neoplastic cells. Methods of assaying cell growth and proliferation are known in the art and are described herein. (See, for example, Kittler et al. (Nature. 432 (7020):1036-40, 2004) and by Miyamoto et al. (Nature 416(6883):865-9, 2002)). Assays for cell proliferation generally involve the measurement of DNA synthesis during cell replication. In one embodiment, DNA synthesis is detected using labeled DNA precursors, such as ([³H]-thymidine or 5-bromo-2′-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

Candidate compounds that reduce the survival of a neoplastic cell under hypoxic conditions are particularly useful as anti-neoplasm therapeutics. Assays for measuring cell viability are known in the art, and are described, for example, by Crouch et al. (J. Immunol. Meth. 160, 81-8); Kangas et al. (Med. Biol. 62, 33843, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull et al., Heterocyclic Chem. 25, 911, 1988). Assays for cell viability are also available commercially. These assays include CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay.

Candidate compounds that increase neoplastic cell death, particularly under hypoxic conditions, (e.g., increase apoptosis) are also useful as anti-neoplasm therapeutics. Assays for measuring cell apoptosis are known to the skilled artisan. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Treatment of an Ischemic Disease

The overexpression of PDK1, which inhibits PDH, was found to enhance the survival of normal cell subjected to hypoxia. Such conditions typically occur during ischemia. Ischemia results when blood flow to a cell, tissue, or organ is interrupted. Tissue damage related to apoptotic cell death often results. Ischemic diseases are characterized by cell or tissue damage related to hypoxia. Exemplary ischemic diseases include, but are not limited to, ischemic injuries caused by a myocardial infarction, a stroke, a transient ischemic episode, a reperfusion injury, physical injury, renal failure, a secondary exsanguination, or blood flow interruption resulting from any other primary diseases. The effects of ischemia are particularly devastating in the brain, when stroke, traumatic brain injury, myocardial infarction, or a transient ischemic attack limits blood flow to the tissues of the CNS. If the interruption of blood flow effects a large area of the CNS, or lasts for a long period of time, death due to loss of neurological function required for viability occurs. If blood flow to the CNS is transiently interrupted and recirculation is established within minutes, only certain neurons in the brain will die. Accordingly, the invention provides therapeutic and prophylactic compositions (e.g., fluoropyruvate) useful for the treatment of ischemia.

The blood-brain barrier limits the uptake of many therapeutic agents into the brain and spinal cord from the general circulation. Molecules which cross the blood-brain barrier use two main mechanisms: free diffusion and facilitated transport. Because of the presence of the blood-brain barrier, attaining beneficial concentrations of a given therapeutic agent in the CNS may require the use of specific drug delivery strategies. Delivery of therapeutic agents to the CNS can be achieved by several methods. One method relies on neurosurgical techniques. In the case of gravely ill patients, surgical intervention is warranted despite its attendant risks. For instance, therapeutic agents can be delivered by direct physical introduction into the CNS, such as intraventricular, intralesional, or intrathecal injection. In traventricular injection can be facilitated by an intraventricular catheter, for example, attached to a reservoir, such as an Ommaya reservoir. Methods of introduction are also provided by rechargeable or biodegradable devices. Another approach is the disruption of the blood-brain barrier by substances which increase the permeability of the blood-brain barrier. Examples include intra-arterial infusion of poorly diffusible agents such as mannitol, pharmaceuticals which increase cerebrovascular permeability such as etoposide, or vasoactive agents such as leukotrienes.

In addition, the invention provides methods of screening for compounds that increase the biological activity or expression of PDK or that inhibit the biological activity or expression of PDH. Such compounds are useful for enhancing the survival of cells at risk of cell death associated with hypoxia. In one embodiment, compounds that inhibit PDH or that enhance the biological activity or expression of PDK are evaluated in tissues or cells treated with the compound under hypoxic conditions relative to untreated control samples. Cell survival is then measured using standard methods. Compounds that enhance the survival of a normal cell under hypoxic conditions are identified as useful in the methods of the invention.

Compounds that inhibit PDH biological activity or expression or that enhance a PDK biological activity or expression may be used to protect cells, tissues, and organs from damage by enhancing the survival of cells at risk of hypoxic cell death. Individuals at increased risk of an ischemic disease due to a hereditary condition are also candidates for such treatment.

### Screening Assays

Compositions of the invention are useful for the high-throughput low-cost screening of candidate compounds that are useful for reducing the survival of a neoplastic cell or for enhancing the survival of a cell at risk of cell death related to hypoxia. Any number of methods are available for carrying out screening assays to identify new candidate compounds. In one embodiment, a compound that promotes an increase in cell survival or a reduction in apoptosis related to hypoxia is considered useful in the invention; such a candidate compound may be used, for example, as a therapeutic to prevent, delay, ameliorate, stabilize, or treat the toxic effects of hypoxia on a cell at risk of cell death. In other embodiments, the candidate compound prevents, delays, ameliorates, stabilizes, or treats a disease or disorder characterized by hypoxic cell death (e.g., an ischemic disease) or promotes the survival of a cell, tissue, or organ at risk of hypoxic cell death, such as a cardiac cell or neuronal cell. Such therapeutic compounds are useful in vivo.

In one example, candidate compounds are screened for those that specifically bind to a PDK or PDH polypeptide or fragment thereof. The efficacy of such a candidate compound is dependent upon its ability to interact with the PDK or PDH polypeptide, or with functional equivalents thereof. Such an interaction can be readily assayed using any number of standard binding techniques and functional assays (e.g., those described in Ausubel et al., supra). In one embodiment, a compound that binds PDK is assayed in a neoplastic cell in vitro for the ability to inhibit PDK activity and reduce neoplastic cell survival. In another embodiment, a compound that interacts with PDH is evaluated for its ability to enhance the survival of a cell at risk of cell death related to hypoxia. The ability of the compound to promote cell survival depends on the ability of the compound to interact with PDH.

In another example, a candidate compound that binds to PDH or PDK is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for PDH or PDK is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds and chimeric polypeptides identified using such methods are then assayed for their effect on cell survival as described herein.

In yet another example, the compound, e.g., the substrate, is coupled to a radioisotope or enzymatic label such that binding of the compound to the substrate, (e.g., the PDH, PDK1, PDK2, PDK3, PDK4) can be determined by detecting the labeled compound, e.g., substrate, in a complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which a PDH or PDK polypeptide or a biologically active portion thereof is contacted with a test compound and the ability of the test compound to bind to the polypeptide thereof is evaluated.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule; which in turn is able to fluoresce due to the absorbed energy. Alternately, the ‘donor’ protein molecule may simply utilize the natural fluorescent energy of trptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to a PDH or PDK1 polypeptide can be accomplished using real-time Biomolecular Interaction Analysis (BIA) (see, e.g., Sjolander S. and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

It may be desirable to immobilize either the candidate compound or its PDH or PDK target to facilitate separation of complexed from uncomplexed forms of one or both of the proteins, as well as to accommodate automation of the assay. Binding of a candidate compound to a PDH or PDK polypeptide, or interaction of a test compound with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/PDH or PDK polypeptide fusion proteins can be adsorbed onto glutathione sepharose beads (Sigma Chemical, St. Louis, Mo.) or glutathione derivatized microtiter plates, which are then combined with the test compound or the test compound and a sample comprising the GST-tagged PDH or PDK1 polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Other techniques for immobilizing a complex of a test compound and a PDH or PDK polypeptide on matrices include using conjugation of biotin and streptavidin. For example, biotinylated proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, an anti-PDH or PDK antibody is identified that reacts with an epitope on the PDH or PDK polypeptide. Methods for detecting binding of a PDH or PDK antibody to the receptor are known in the art and include immunodetection of complexes, as well as enzyme-linked assays which rely on detecting an enzymatic activity associated with the channel. Antibodies that bind a PDH or PDK polypeptide are then tested for the ability to inhibit the polypeptide. Such antibodies or compounds that bind a PDK polypeptide may be tested for their activity in reducing the survival of a neoplastic cell, including a hypoxic neoplastic cell, as described herein. Alternatively, antibodies or compounds that bind a PDH polypeptide may be tested for their activity in promoting the survival of a cell at risk of cell death related to hypoxia.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of a PDH or PDK1 polypeptide, e.g., by including a membrane component or synthetic membrane components.

In a specific embodiment, the assay includes contacting the PDH or PDK polypeptide or a biologically active portion thereof with a known compound which binds the PDH or PDK polypeptide to form an assay mixture, contacting the assay mixture with a test compound, and determining the ability of the test compound to interact with a PDH or PDK polypeptide, wherein determining the ability of the test compound to interact with a PDH or PDK polypeptide includes determining the ability of the test compound to preferentially bind to the PDH or PDK polypeptide, or to modulate the activity of the PDH or PDK polypeptide, as compared to the known compound.

Compounds isolated by this method (or any other appropriate method) may, if desired, be further purified (e.g., by high performance liquid chromatography). In addition, these candidate compounds may be tested for their ability to increase the activity of a PDH or PDK polypeptide (e.g., as described herein). Compounds that bind an inhibit PDH isolated by this approach may also be used, for example, as therapeutics to treat ischemic cell death in a subject. Compounds that bind an inhibit PDK isolated by this approach may also be used, for example, as therapeutics to treat neoplastic cell death related to hypoxia. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

In another embodiment, a candidate compound is tested for its ability to enhance the biological activity of a PDK polypeptide. The biological activity of a PDK polypeptide is assayed using any standard method. For example, PDK biological activity is assayed by measuring kinase activity, such as by measuring the phosphorylation state of a PDK substrate (e.g., PDH).

In another embodiment, a PDK or PDH nucleic acid described herein is expressed as a transcriptional or translational fusion with a detectable reporter, and expressed in an isolated cell (e.g., mammalian or insect cell) under the control of an endogenous or a heterologous promoter. The cell expressing the fusion protein is then contacted with a candidate compound, and the expression of the detectable reporter in that cell is compared to the expression of the detectable reporter in an untreated control cell. A candidate compound that decreases the expression of the PDK detectable reporter is a compound that is useful for the treatment of a neoplasia. A candidate compound that decreases the expression of a PDH detectable reporter is a compound that is useful for the treatment or prevention of an ischemic disease. In preferred embodiments, the candidate compound decreases the expression of a reporter gene fused to a PDH or PDK nucleic acid molecule.

One skilled in the art appreciates that the effects of a candidate compound on PDH or PDK expression or biological activity are typically compared to the expression or activity of PDH or PDK in the absence of the candidate compound. Thus, the screening methods include comparing the value of a cell modulated by a candidate compound to a reference value of an untreated control cell.

Expression levels can be compared by procedures well known in the art such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, and ELISA, microarray analysis, or colorimetric assays, such as the Bradford Assay and Lowry Assay,

Changes in neoplastic cell growth or ischemic damage further comprise values and/or profiles that can be assayed by methods of the invention by any method known in the art, including x-ray, sonogram, ultrasound, MRI, or PET scan.

Molecules that alter PDH or PDK expression or activity include organic molecules, peptides, peptide mimetics, polypeptides, nucleic acids, and antibodies that bind to a PDH or PDK nucleic acid sequence or polypeptide and alter its expression or biological activity are preferred.

Each of the DNA sequences listed herein may also be used in the discovery and development of a therapeutic compound for the treatment of a neoplasia or an ischemic disease. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of altering the activity of a PDH or PDK polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrinack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds maybe presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to increase the activity of a PDH or PDK polypeptide, or binding to a PDH or PDK polypeptide, further fractionation of the positive lead extract is necessary to isolate chemical constituents responsible for the observed effect. Thus, the goal of the extraction, fractionation, and purification process is the careful characterization and identification of a chemical entity within the crude extract that alter the activity of a PDH or PDK polypeptide. Methods of fractionation and purification of such heterogenous extracts are known in the art. If desired, compounds shown to be useful as therapeutics for the treatment of a neoplasia or an ischemic disease are chemically modified according to methods known in the art.

If desired, candidate compounds selected using any of the screening methods described herein are tested for their efficacy using animal models of neoplasia. In one approach, the effect of a candidate compound on tumor load is analyzed in mice injected with human neoplastic cells. The neoplastic cell is allowed to grow to form a mass, preferably a hypoxic cell mass. The mice are then treated with a candidate compound or vehicle (PBS) daily for a period of time to be empirically determined. Mice are euthanized and the neoplastic tissue is collected. The mass of the neoplastic tissue in mice treated with the selected candidate compounds is compared to the mass of neoplastic tissue present in corresponding control mice.

In another approach, mice are injected with neoplastic human cells. The mice containing the neoplastic cells are then injected (e.g., intraperitoneally) with vehicle (PBS) or candidate compound daily for a period of time to be empirically determined. Mice are then euthanized and the neoplastic tissues are collected and analyzed for PDK or PDH nucleic acid or protein levels using methods described herein. Compounds that decrease PDK mRNA or protein expression relative to control levels are expected to be efficacious for the treatment of a neoplasm in a subject (e.g., a human patient).

Preferably, compounds selected according to the methods of the invention reduce the growth, proliferation, or severity of the neoplasm by at least 10%, 25%, or 50%, or by as much as 75%, 85%, or 95% when compared to a control.

In another approach, a compound identified according to the methods described herein as useful for the treatment of ischemia is tested in an animal model of ischemia. In one approach, a candidate compound is provided to a mouse before, during, or after the induction of ischemia in a selected tissue (e.g., heart, brain, hind limb). The level of tissue damage in the selected tissue is then compared to the damage present in a corresponding tissue in a control animal that did not receive the candidate compound. Compounds that reduce the level of tissue damage (e.g., promote cell survival, reduce apoptosis) are identified as useful in the methods of the invention. Animal models of ischemia are known in the art and are described for example, by Maloyan et al. (Physiol Genomics. 2005 Sep. 21; 23(1):79-88), which describes a model of cardiac ischemia; by Patel (Cardiovasc Res. 2005 Oct. 1; 68(1):144-54), which describes a model of limb ischemia; and by Comi et al, (Pediatr Neurol. 31:254-7, 2004), which describes a stroke and ischemic seizure model.

### Recombinant Polypeptide Expression

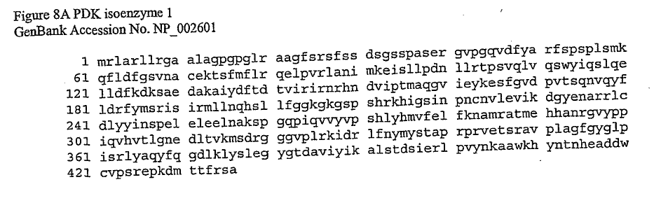

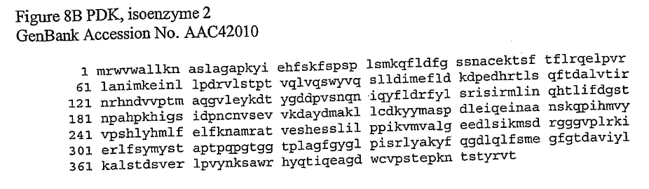

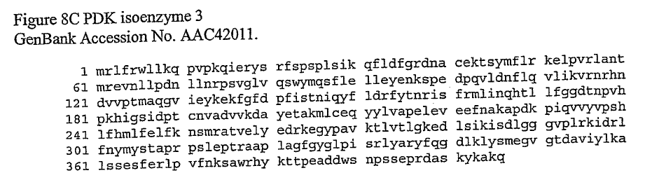

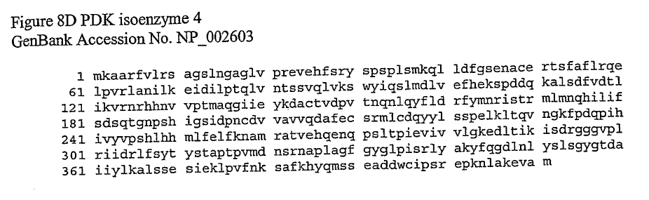

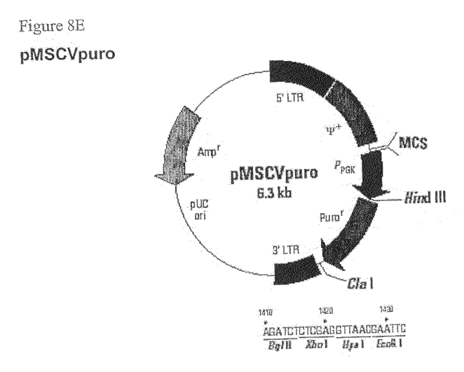

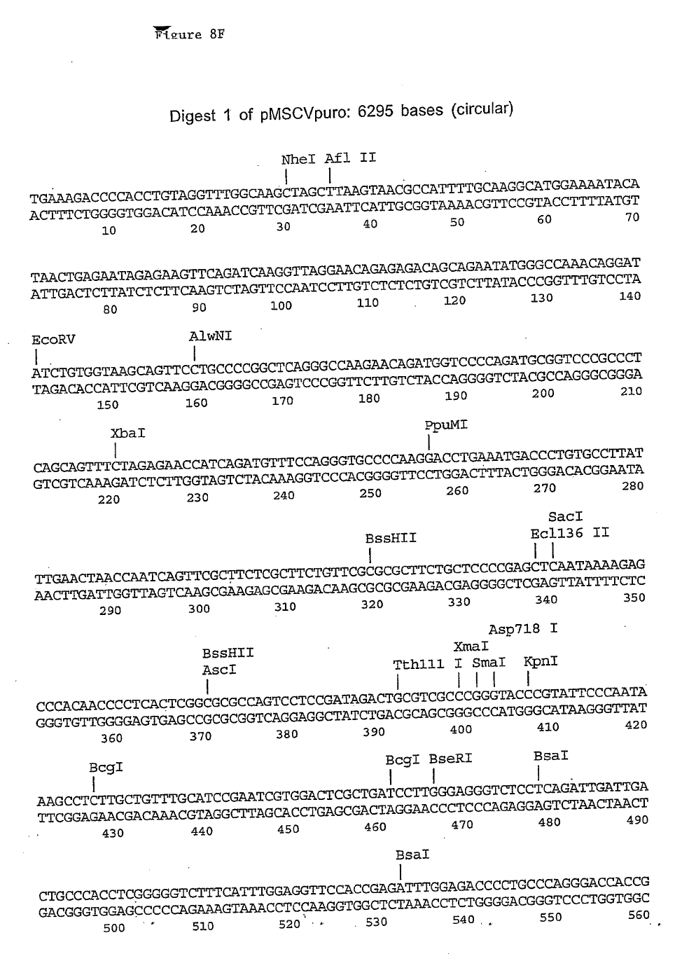

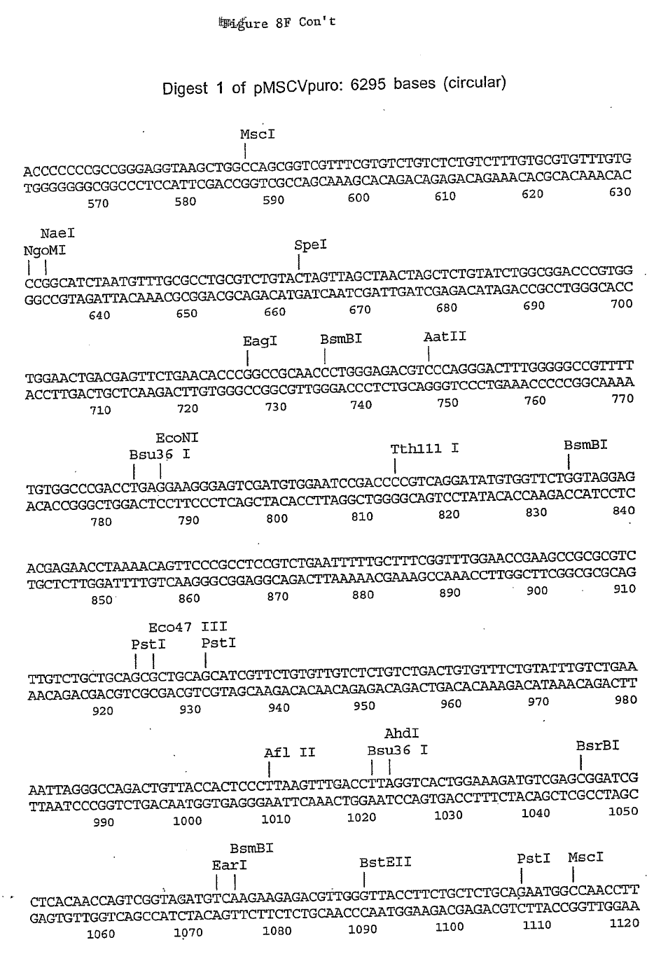

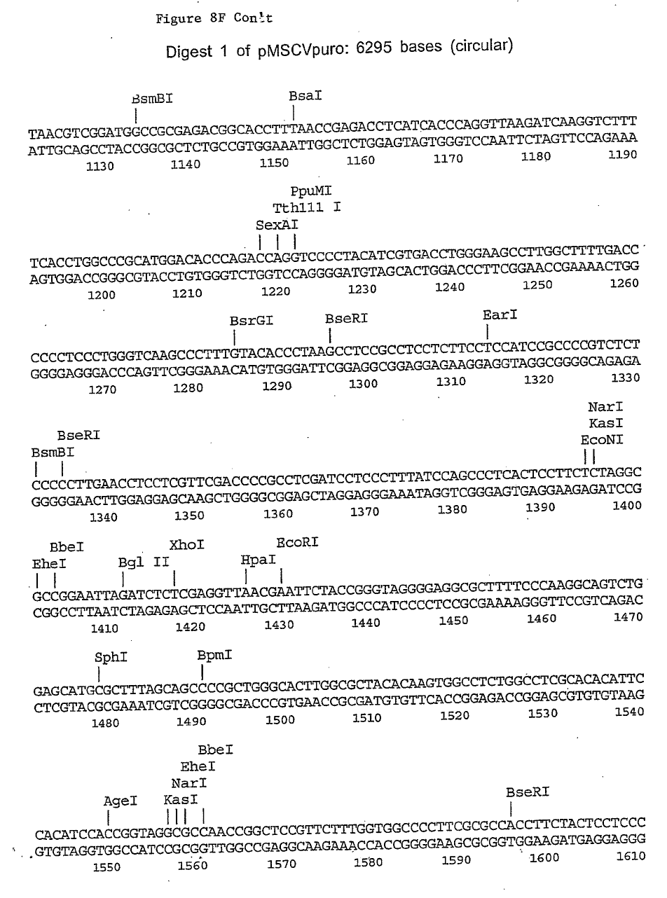

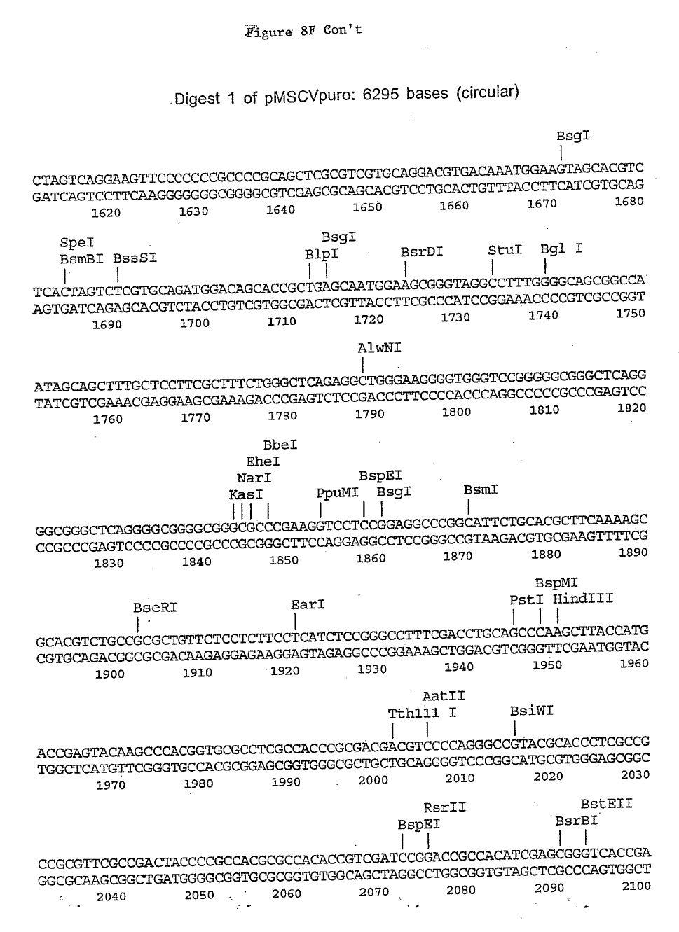

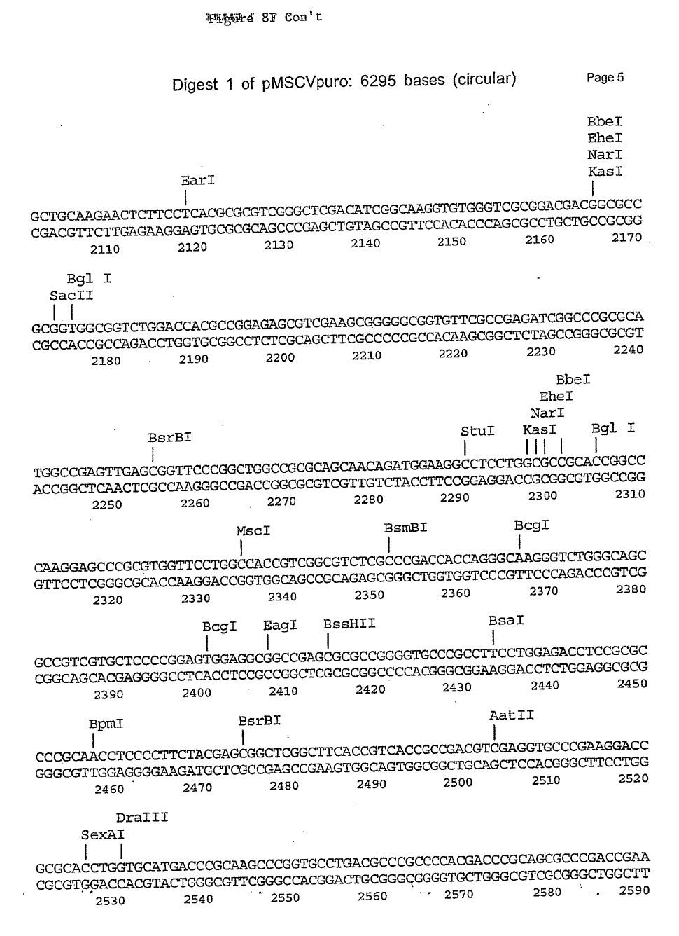

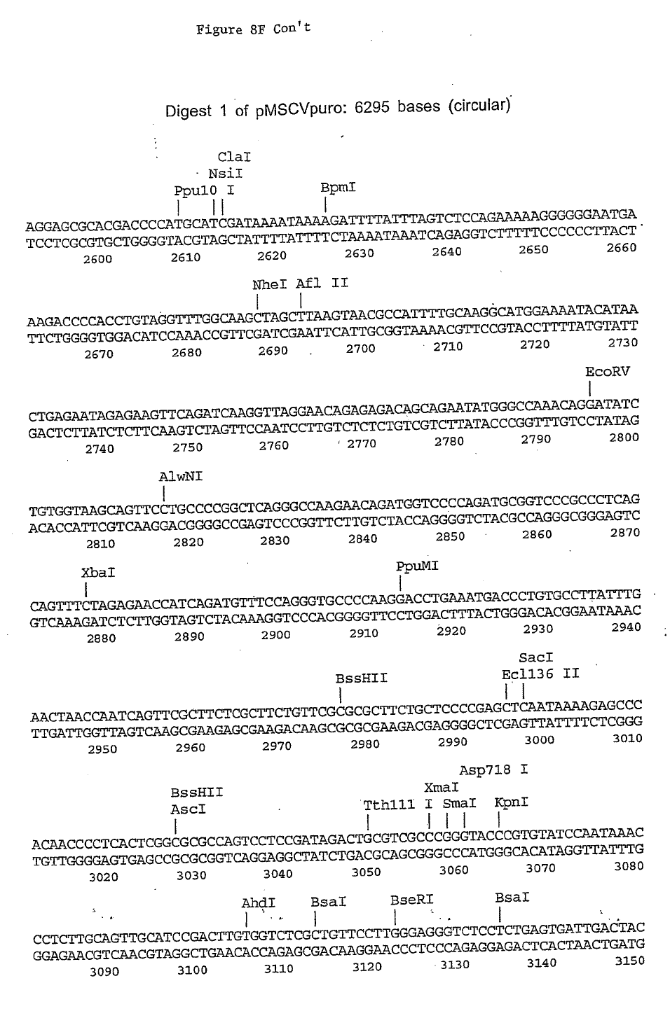

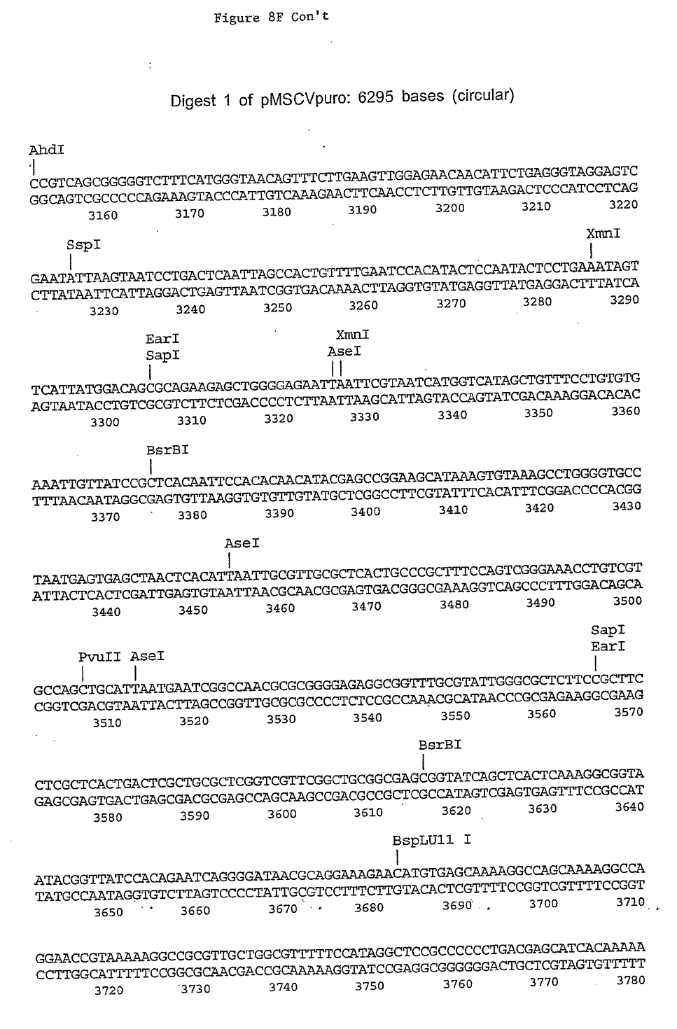

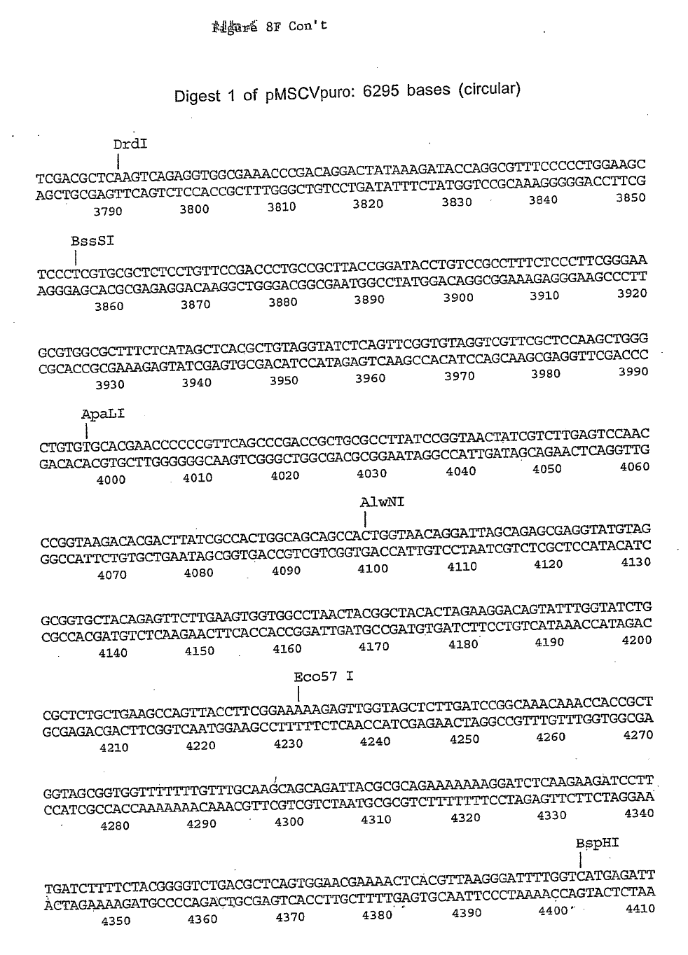

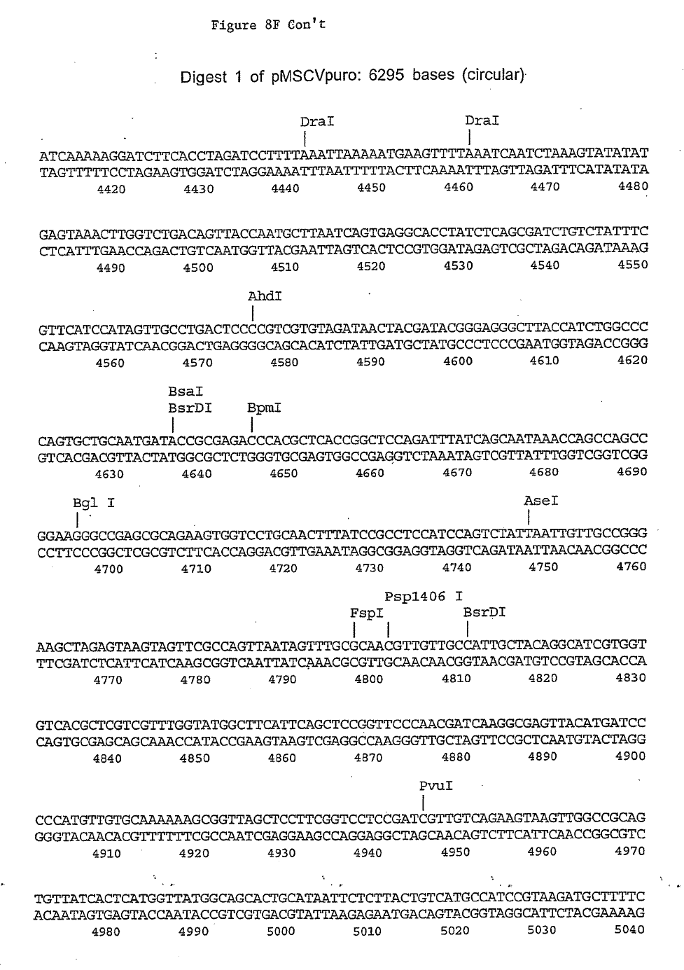

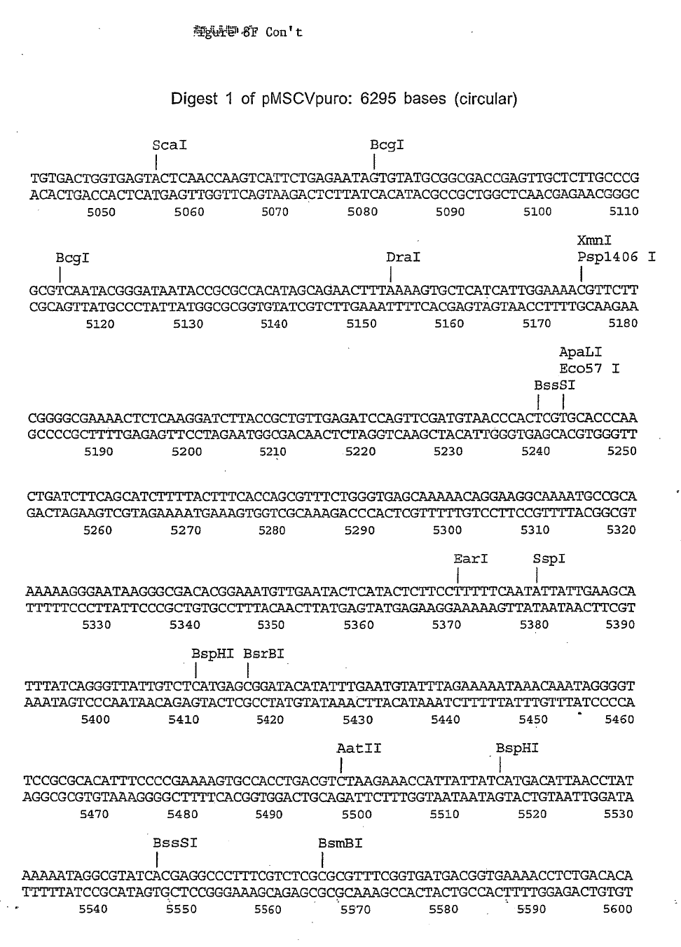

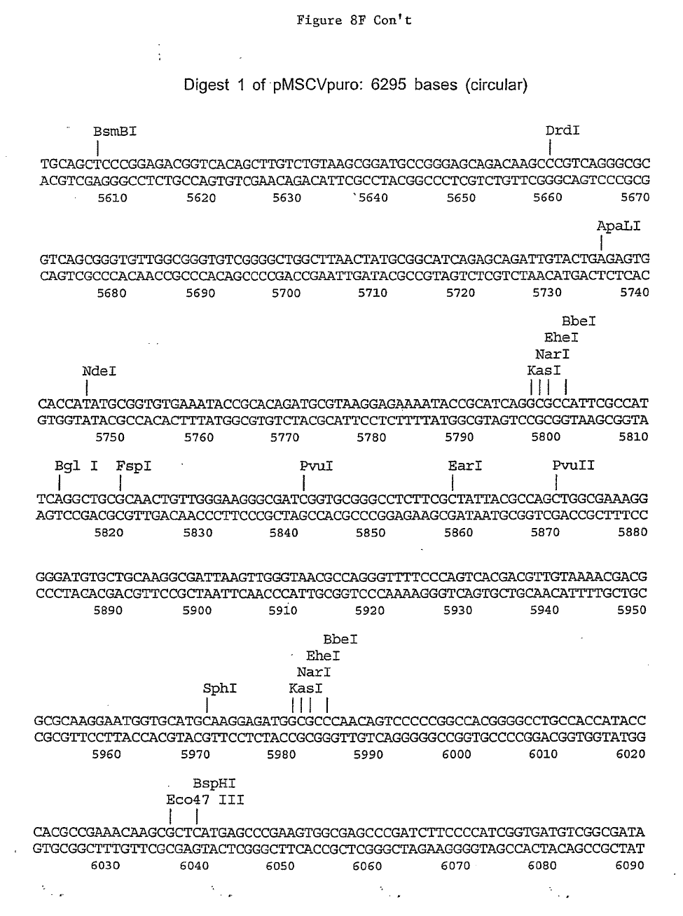

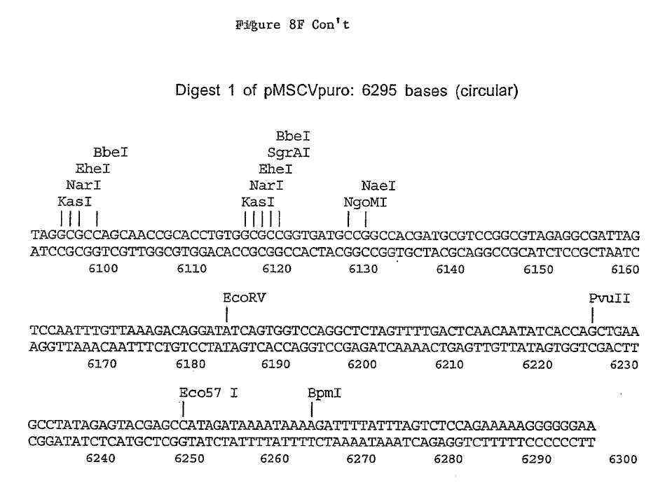

Compound screening is facilitated by the availability of large quantities of purified PDK or PDH polypeptides that are recombinantly expressed. In general, recombinant polypeptides of the invention may be produced by transformation of a suitable host cell with all or part of a polypeptide-encoding nucleic acid molecule or fragment thereof in a suitable expression vehicle. The amino acid sequence of PDK1 is provided at GenBank Accession No. NM_{—}002610; PDK2 is provided at GenBank Accession No. AAC42010; PDK3 is provided at GenBank Accession No. AAC42011; PDK4 is provided at GenBank Accession No NP_{—}002603. The sequence of pyruvate dehydrogenase alpha 1 is provided at GenBank Accession No NM_{—}000284. Select sequences useful in the methods of the invention are shown in FIGS. 8A-8F.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984. The Pierce Chemical Co., Rockford, Ill.). These general techniques of polypeptide expression and purification can also be used to produce and isolate useful peptide fragments or analogs (described herein).

### PDK1 Polypeptides and Analogs

Overexpression of a PDK1 polypeptide or fragment thereof promotes the survival of cells at risk of hypoxic cell death. Included in the invention are PDK1, PDK2, PDK3 and PDK4 analogs, or fragments thereof, that are modified in ways that enhance their ability to promote the survival of a cell at risk of hypoxic cell death. In one embodiment, the invention provides methods for optimizing a PDK amino acid sequence or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also includes fragments of any one of the polypeptides of the invention. As used herein, the term “a fragment” means at least 10, 25, 50, 75, 100, 150, or 200 amino acids. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein PDK analogs having a chemical structure designed to mimic PDK functional activity can be administered according to methods of the invention. PDK analogs may exceed the physiological activity of the original polypeptide. Methods of analog design are well known in the art, and synthesis of analogs can be carried out according to such methods by modifying the chemical structures such that the resultant analogs exhibit the cell death modulating activity of a reference PDK chimeric polypeptide. These chemical modifications include, but are not limited to, substituting alternative R groups and varying the degree of saturation at specific carbon atoms of a reference PDK polypeptide. Preferably, the PDK analogs are relatively resistant to in vivo degradation, resulting in a more prolonged therapeutic effect upon administration. Assays for measuring functional activity include, but are not limited to, those described in the Examples below.

### Inhibitory Nucleic Acid Molecules

Inhibitory nucleic acid molecules (e.g., siRNAs, shRNAs, antisense) are useful for reducing the expression of a PDK or PDH. Accordingly, the invention provides inhibitory nucleic acid molecules that are useful for decreasing the expression of a polypeptide of interest (e.g., PDK1, PDK2, PDK3, PDK4 or PDH). Inhibitory nucleic acid molecules include, but are not limited to double-stranded RNAs, antisense RNAs, and siRNAs, or portions thereof. As reported in more detail below, the inhibition of PDK1 expression by an siRNA reduced the survival of neoplastic cells under hypoxic conditions.

The inhibitory nucleic acids of the present invention may be employed in double-stranded RNAs for RNA interference (RNAi)-mediated knock-down of PDK1, PDK2, PDK3, PDK4, or PDH expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). RNA interference (RNAi) provides for the targeting of specific mRNAs for degradation by complementary short-interfering RNAs (siRNAs). RNAi is a useful therapeutic approach for gene silencing. The general mechanism of RNAi involves the cleavage of double-stranded RNA (dsRNA) to short 21-23-nt siRNAs. This processing event is catalyzed by Dicer, a highly conserved, dsRNA-specific endonuclease that is a member of the RNase III family. Processing by Dicer results in siRNA duplexes that have 5′-phosphate and 3′-hydroxyl termini, and subsequently, these siRNAs are recognized by the RNA-induced silencing complex (RISC). Active RISC complexes (RISC*) promote the unwinding of the siRNA through an ATP-dependent process, and the unwound antisense strand guides RISC* to the complementary mRNA. The targeted mRNA is then cleaved by RISC* at a single site that is defined with regard to where the 5′-end of the antisense strand is bound to the mRNA target sequence. siRNAs use as therapeutic agents is improved by modifications that enhance the stability of siRNAs.

In one embodiment of the invention, a double-stranded RNA (dsRNA) molecule includes between eight and twenty-five consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Given the sequence of a mammalian gene (e.g., PDK1, PDK2, PDK3, PDK4, or PDH), siRNAs may be designed to inactivate that gene. For example, for a gene that consists of 2000 nucleotides, approximately 1,978 different twenty-two nucleotide oligomers could be designed; this assumes that each oligomer has a two base pair 3′ overhang, and that each siRNA is one nucleotide residue from the neighboring siRNA. To effectively silence the gene, only a few of these twenty-two nucleotide oligomers would be needed; approximately 1, 5, 10, or 12 siRNAs could be sufficient to significantly reduce mammalian gene activity. In one embodiment, an siRNA that targets PDH or PDK is transferred into a mammalian cell in culture, and the effect of the siRNAs on the PDK or PDH expression or activity in the cultured cells is assayed. Methods for assaying PDK activity are known in the art (Aicher et al., supra; Mann et al., supra) and are described herein. Methods for assaying PDH activity are described, for example, by Aicher et al. (J. Med. Chem. 43:236-249, 2000). Alternatively, siRNAs could be injected into an animal, for example, into the blood stream (McCaffrey et al., Nature 418:38-92002).

Unmodified siRNAs may be limited in their therapeutic applications by their sensitivity towards nucleases. Chemical strategies to improve stability such as the modification of the deoxyribo/ribo sugar and the heterocyclic base are known in the art, as are the modification or replacement of the internucleotide phosphodiester linkage. Methods for enhancing siRNA stability are described, for example, by Chiu et al., (RNA 9:1034-1048, 2003); Layzer, et al. (RNA 10, 766-771, 2004); and by Morrissey et al., (Nature Biotechnology 23, 1002-1007, 2005). In various approaches, fully modified 2′-O-propyl and 2′-O-pentyl oligoribonucleotides are used to enhance inhibitory nucleic acid stability chemical modifications that stabilized interactions between A-U base pairs; thioate linkages (P-S) are integrated into the backbone; uridine and cytidine in the antisense strand of siRNA are replaced with 2′-fluoro-uridine (2′-FU) and 2′-fluoro-cytidine (2′-FC), respectively, which have a fluoro group at the 2′-position in place of the 2′-OH; 5-bromo-uridine (U[5Br]), 5-iodo-uridine (U[5I]), or 2,6-diaminopurine (DAP) are included in the siRNA. Such approaches are useful for enhancing siRNA stability. Other useful modifications for enhancing siRNA stability are described below.

In another approach, antisense oligonucleotides are used to decrease the expression of PDH or PDK. The efficacy of antisense technology lies in the specific binding of an oligoribonucleotide to its target sequence. The formation of a duplex between an antisense oligomer and its target sequence prevents gene expression by interfering with subsequent processing, transport or translation, or by degradation of the RNA via RNase H. As for siRNA, the therapeutic efficacy of antisense molecules is improved by modifications that enhance the stability of the antisense molecule.

### Modifications to Enhance Inhibitory Nucleic Acid Molecule Stability

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred inhibitory nucleic acid molecules useful in this invention include oligonucleotides containing modified backbones or non-natural internucleoside linkages. As defined in this specification, inhibitory nucleic acid molecules having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be inhibitory nucleic acid molecules.

Inhibitory nucleic acid molecules that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Inhibitory nucleic acid molecules having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other inhibitory nucleic acid molecules, both the sugar and the internucleoside linkage, i.e., the backbone, are replaced with novel groups. One such inhibitory nucleic acid molecules, is referred to as a Peptide Nucleic Acid (PNA). In PNA compounds, the sugar-backbone of an oligonucleotide is replaced with an amide containing backbone, in particular an aminoethylgiycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O— N(CH₃)—CH₂—CH₂—. In other embodiments, the oligonucleotides have morpholino backbone structures described in U.S. Pat. No. 5,034,506.

Inhibitory nucleic acid molecules may also contain one or more substituted sugar moieties. inhibitory nucleic acid molecules comprise one of the following at the 2′ position: OH; F; O—, S—, or N-alkyl; O—, S—, or N-alkenyl; O—, S— or N—alkynyl; or O-alkyl-O-alkyl, wherein the alkyl, alkenyl, and alkynyl may be substituted or unsubstituted C₁ to C₁₀ alkyl or C₂to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH.sub.3, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′—OCH₂CH.₂CH₂NH₂) and 2′-fluoro (2′—F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Inhibitory nucleic acid molecules may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Inhibitory nucleic acid molecules may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thiolalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcrytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of an inhibitory nucleic acid of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556,-1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes inhibitory nucleic acid molecules that are chimeric compounds. “Chimeric” inhibitory nucleic acid molecules are inhibitory nucleic acid molecules, particularly oligonucleotides, that contain two or more chemically distinct regions, each made up of at least one monomer unit, i.e., a nucleotide in the case of an oligonucleotide. These ₂ typically contain at least one region where the nucleobase oligomer is modified to confer, upon the ₂, increased resistance to nuclease degradation, increased cellular uptake, and/or increased binding affinity for the target nucleic acid. An additional region of the inhibitory nucleic acid molecule, such as an antisense molecule, may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter inhibitory nucleic acid molecules when chimeric inhibitory nucleic acid molecules are used, compared to phosphorothioate deoxyoligonucleotides hybridizing to the same target region.

Chimeric inhibitory nucleic acid molecules of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The inhibitory nucleic acid molecules used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The inhibitory nucleic acid molecules of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The inhibitory nucleic acid molecules of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

### PDH or PDK Antibodies

Antibodies are well known to those of ordinary skill in the science of immunology. Particularly useful in the methods of the invention are antibodies that specifically bind a PDK or PDH polypeptide and inhibit the activity of the polypeptide. Antibodies that inhibit the activity of PDK are useful for the treatment of a neoplasia while antibodies that inhibit PDH activity are useful for the treatment of an ischemic disease. Accordingly, an antibody that specifically binds PDH or PDK is assayed for such activity as described herein.

As used herein, the term “antibody” means not only intact antibody molecules, but also fragments of antibody molecules that retain immunogen binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of 60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making and using unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

In one embodiment, an antibody that binds an PDH or PDK polypeptide is monoclonal. Alternatively, the anti-PDH or PDK antibody is a polyclonal antibody. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fe′ region, designated an “F(ab′)₂” fragment, retains both of the antigen binding sites of the intact antibody. Similarly, an antibody from which the Fc region has been enzymatically cleaved, or which has been produced without the Fc region, designated an “Tab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing PDH or PDK1 polypeptides, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding an PDH or PDK polypeptide, or immunogenic fragments thereof, can be provided to the host in a delivery vehicle that is taken up by immune cells of the host. The cells will in turn express the receptor on the cell surface generating an immunogenic response in the host. Aternatively, nucleic acid sequences encoding an PDH or PDK polypeptide, or immunogenic fragments thereof, can be expressed in cells in vitro, followed by isolation of the receptor and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed. for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

### Pharmaceutical Therapeutics

The invention provides a simple means for identifying compositions (including nucleic acids, peptides, small molecule inhibitors, and mimetics) capable of acting as therapeutics for the treatment of a neoplasia or an ischemic disease. Using the methods of the invention, dichloroacetate, which inhibits pyruvate dehydrogenase kinase, was identified as a compound that inhibits the ability of neoplastic cells to survive hypoxia. Using the methods described herein, other compounds having the ability to inhibit PDK and reduce the survival of a neoplastic cell may be identified. In addition, the invention provides for the identification of compounds that inhibit PDH and enhance the survival of a cell at risk of hypoxic cell death. A compound discovered to have medicinal value using the methods described herein is useful as a drug or as information for structural modification of existing compounds, e.g., by rational drug design. Such methods are useful for screening compounds having an effect on the expression or activity of a PDH or PDK polypeptide.

For therapeutic uses, the compositions or agents identified using the methods disclosed herein may be administered systemically, for example, formulated in a pharmaceutically-acceptable buffer such as physiological saline. For the treatment of cancer, the compounds of the invention are preferably delivered systemically by intravenous injection, although intra-arterial delivery may be preferred for the treatment of a liver cancer. For the treatment of ischemia, compounds of the invention are delivered systemically by intravenous injection, although intra-arterial delivery may also be used.

Other routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a neoplasia or ischemic disease therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia or ischemic disease. Generally, amounts will be in the range of those used for other agents used in the treatment of other diseases associated with neoplasia or ischemic disease, although in certain instances lower amounts will be needed because of the increased specificity of the compound. A compound is administered at a dosage that controls the clinical or physiological symptoms of an neoplasia or ischemic disease as determined by a diagnostic method known to one skilled in the art, or using any that assay that measures the expression or the biological activity of a PDH or PDK polypeptide.

In one embodiment, the present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a neoplastic or ischemic disease, disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which PDK or PDH may be implicated.

### Formulation of Pharmaceutical Compositions

The administration of a compound for the treatment of neoplasia or ischemic disease may be by any suitable means that results in a concentration of the therapeutic that, combined with other components, is effective in ameliorating, reducing, or stabilizing an neoplasia or ischemic disease. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a neoplasia or ischemic disease by using carriers or chemical derivatives to deliver the therapeutic agent to a particular cell type (e.g., neoplastic cell or a neuronal or cardiac cell at risk of cell death) whose function is perturbed in neoplasia or ischemic disease. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the neoplasia or ischemic disease therapeutic (s), the composition may include suitable parenterally acceptable carriers and/or excipients. The active neoplasia or ischemic disease therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active neoplasia or ischemic disease therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam- nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms For Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active active neoplasia or ischemic disease therapeutic substance). The coating may be applied on the solid dosage form in a similar manner as that described in Encyclopedia of Pharmaceutical Technology, supra.

At least two active neoplasia or ischemic disease therapeutics may be mixed together in the tablet, or may be partitioned. In one example, the first active therapeutic is contained on the inside of the tablet, and a second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, rmicrocrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active neoplasia or ischemic disease therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, camauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosage Determination

Those of skill in the art will recognize that the best treatment regimens for using compounds of the present invention (e.g., inhibitors of a PDK or PDH) to treat a neoplasia or ischemic disease can be straightforwardly determined. This is not a question of experimentation, but rather one of optimization, which is routinely conducted in the medical arts. In vivo studies in nude mice often provide a starting point from which to begin to optimize the dosage and delivery regimes. The frequency of injection will initially be once a week, as has been done in some mice studies. However, this frequency might be optimally adjusted from one day to every two weeks to monthly, depending upon the results obtained from the initial clinical trials and the needs of a particular patient.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 mg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg compound/Kg body to about 20 mg compound/Kg body. In other embodiments the doses maybe about 8, 10, 12, 14, 16 or 18 mg/Kg body weight. In one preferred approach, a compound identified as useful for the treatment of a neoplasia is administered to achieve a serum concentration between 25 and 250 nM (e.g., 25 nM, 50 nM, 75 nM 100 nM, 125 nM, 150 nM, 200 nM, or 250 nM). Of course, this dosage amount may be adjusted upward or downward, as is routinely done in such treatment protocols, depending on the results of the initial clinical trials and the needs of a particular patient.

### PDK Polynucleotide Therapy

As described herein, cell death related to hypoxia, such as cell death associated with ischemia, transient ischemic attacks, reperfusion injury, traumatic injury, stroke, and myocardial infarction, can be inhibited by the over-expression of PDK. Therefore, polynucleotide therapy featuring a polynucleotide encoding a PDK protein, variant, or fragment thereof is one therapeutic approach for treating an ischemic disease (e.g., ischemia, transient ischemic attacks, reperfusion injury, traumatic injury, stroke, and myocardial infarction). Such nucleic acid molecules can be delivered to cells of a subject having or susceptible to ischemia. The nucleic acid molecules must be delivered to the cells of a subject in a form in which they can be taken up so that therapeutically effective levels of an PDK protein or fragment thereof can be produced.

Transducing viral (e.g., retroviral, adenoviral, and adeno-associated viral) vectors can be used for somatic cell gene therapy, especially because of their high efficiency of infection and stable integration and expression (see, e.g.; Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an PDK protein, variant, or a fragment thereof, can be cloned into a retroviral vector and expression can be driven from its endogenous promoter, from the retroviral long terminal repeat, or from a promoter specific for a target cell type of interest (e.g., in a cardiac cell or in a neuronal cell).

Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cometta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer an PDK polynucleotide systemically or to a cell or tissue of interest (e.g., a cardiac cell or neuronal cell).

Non-viral approaches can also be employed for the introduction of therapeutic to a cell of a patient having or at risk of developing cellular damage related to ischemia (e.g., ischemia, transient ischemic attacks, reperfusion injury, traumatic injury, stroke, and myocardial infarction). For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known-to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant PDK protein, variant, or fragment thereof, either directly to the site of a potential or actual disease-affected tissue or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Patient Monitoring

The disease state or treatment of a patient having a neoplasia can be monitored using the methods and compositions of the invention. In one embodiment, the expression or activity of a PDK nucleic acid molecule or polypeptide is monitored using any method known in the art. In another embodiment, phosphorylated PDH is assayed. Neoplastic cells that have acquired mutations that permit their survival under hypoxic conditions are particularly aggressive, and therefore require more aggressive treatment regiments. Accordingly, an increase in the expression of PDK1 or an increase in phosphorylated PDH in a patient sample identifies the neoplasia as particularly severe. Therapeutics that decrease the expression of a PDK1 nucleic acid molecule or polypeptide or a decrease in phosphorylated PDH are taken as particularly useful in the invention. Such monitoring may be useful, for example, in assessing the efficacy of a particular drug in a patient or in assessing patient compliance with a treatment regimen.

### Kits

The invention provides kits for the treatment or prevention of a neoplasia or an ischemic disease, or symptoms thereof. In one embodiment, the kit includes a PDK inhibitor for use in neoplasia or a PDH inhibitor of PDK expression vector for use in ischemia. In some embodiments, the kit comprises a sterile container which contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired compositions of the invention are provided together with instructions for administering them to a subject having or at risk of developing a neoplasia or ischemia. The instructions will generally include information about the use of the compositions for the treatment or prevention of a neoplasia or ischemia. In other embodiments, the instructions include at least one of the following: description of the composition; dosage schedule and administration for treatment of a neoplasia, ischemia, or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### Diagnostics

Neoplastic tissues that have acquired the ability to survive under hypoxic conditions express higher levels of PDK polypeptides or polynucleotides, as well as higher levels of phosphorylated PDH than corresponding normal tissues. Accordingly, expression levels of an PDK or phosphorylated PDH are correlated with neoplasia, particularly aggressive neoplasias, and thus are useful in diagnosis. Accordingly, the present invention provides a number of diagnostic assays that are useful for the identification or characterization of a neoplasia.

In one embodiment, a patient having a neoplasia will show an increase in the expression of an PDK nucleic acid molecule. Alterations in gene expression are detected using methods known to the skilled artisan and described herein. Such information can be used to diagnose a neoplasia. In another embodiment, an alteration in the expression of an PDK nucleic acid molecule is detected using real-time quantitative PCR (Q-rt-PCR) to detect changes in gene expression.

Primers used for amplification of an PDK nucleic acid molecule, including but not limited to those primer sequences described herein, are useful in diagnostic methods of the invention. The primers of the invention embrace oligonucleotides of sufficient length and appropriate sequence so as to provide specific initiation of polymerization on a significant number of nucleic acids. Specifically, the term “primer” as used herein refers to a sequence comprising two or more deoxylibonucleotides or ribonucleotides, preferably more than three, and most preferably more than 8, which sequence is capable of initiating synthesis of a primer extension product, which is substantially complementary to a locus strand. The primer must be sufficiently long to prime the synthesis of extension products in the presence of the inducing agent for polymerization. The exact length of primer will depend on many factors, including temperature, buffer, and nucleotide composition. The oligonucleotide primer typically contains between 12 and 27 or more nucleotides, although it may contain fewer nucleotides. Primers of the invention are designed to be “substantially” complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementary with the 5′ and 3′ flanking sequences to hybridize therewith and permit amplification of the genomic locus. While exemplary primers are provided herein, it is understood that any primer that hybridizes with the target sequences of the invention are useful in the method of the invention for detecting PDK1 nucleic acid molecules.

In one embodiment, PDK-specific primers amplify a desired genomic target using the polymerase chain reaction (PCR). The amplified product is then detected using standard methods known in the art. In one embodiment, a PCR product (i.e., amplicon) or real-time PCR product is detected by probe binding. In one embodiment, probe binding generates a fluorescent signal, for example, by coupling a fluorogenic dye molecule and a quencher moiety to the same or different oligonucleotide substrates (e.g., TaqMan® (Applied Biosystems, Foster City, Calif., USA), Molecular Beacons (see, for example, Tyagi et al., Nature Biotechnology 14(3):303-8, 1996), Scorpions® (Molecular Probes Inc., Eugene, Oreg., USA)). In another example, a PCR product is detected by the binding of a fluorogenic dye that emits a fluorescent signal upon binding (e.g., SYBR® Green (Molecular Probes)). Such detection methods are useful for the detection of an PDK1 PCR product.

In another embodiment, hybridization with PCR probes that are capable of detecting an PDK nucleic acid molecule, including genomic sequences, or closely related molecules, may be used to hybridize to a nucleic acid sequence derived from a patient having a neoplasia. The specificity of the probe determines whether the probe hybridizes to a naturally occurring sequence, allelic variants, or other related sequences. Hybridization techniques may be used to identify mutations indicative of a neoplasia, or may be used to monitor expression levels of these genes (for example, by Northern analysis (Ausubel et al., supra).

In yet another embodiment, humans may be diagnosed for a propensity to develop a neoplasia by direct analysis of the sequence of an PDK nucleic acid molecule. The sequence of an PDK nucleic acid molecule derived from a subject is compared to a reference sequence. An alteration in the sequence of the PDK nucleic acid molecule relative to the reference indicates that the patient has or has a propensity to develop a neoplasia.

In another approach, diagnostic methods of the invention are used to assay the expression of an PDK or phosphorylated PDH polypeptide in a biological sample relative to a reference (e.g., the level of PDK1 or phosphorylated PDH polypeptide present in a corresponding control tissue). In one embodiment, the level of an PDK or phosphorylated PDH polypeptide is detected using an antibody that specifically binds one of thoses polypeptides. Such antibodies are useful for the diagnosis of a neoplasia. Methods for measuring an antibody-polypeptide complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra. Immunoassays can be used to determine the quantity of PDK1 or phosphorylated PDH polypeptide in a sample, where an increase in the level of the PDK1 or phosphorylated PDH polypeptide is diagnostic of a patient having a neoplasia.

In general, the measurement of an PDK or phosphorylated PDH polypeptide or nucleic acid molecule in a subject sample is compared with a diagnostic amount present in a reference. A diagnostic amount distinguishes between a neoplastic tissue and a control tissue. The skilled artisan appreciates that the particular diagnostic amount used can be adjusted to increase sensitivity or specificity of the diagnostic assay depending on the preference of the diagnostician. In general, any significant increase (e.g., at least about 10%, 15%, 30%, 50%, 60%, 75%, 80%, or 90%) in the level of an PDK or phosphorylated PDH polypeptide or nucleic acid molecule in the subject sample relative to a reference may be used to diagnose a neoplasia. In one embodiment, the reference is the level of PDK1 or phosphorylated PDH polypeptide or nucleic acid molecule present in a control sample obtained from a patient that does not have a neoplasia. In another embodiment, the reference is a baseline level of PDK or phosphorylated PDH polypeptide present in a biologic sample derived from a patient prior to, during, or after treatment for a neoplasia. In yet another embodiment, the reference is a standardized curve.

### Types of Biological Samples

The level of an PDK or phosphorylated PDH polypeptide polypeptide or nucleic acid molecule can be measured in different types of biologic samples. In one embodiment, the biologic sample is a tissue sample that includes cells of a tissue or organ. Such tissue is obtained, for example, from a biopsy. In another embodiment, the biologic sample is a biologic fluid sample (e.g., blood, blood plasma, serum, urine, seminal fluids, ascites, or cerebrospinal fluid).

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims. along with their full scope of equivalents.

It should be appreciated that the invention should not be construed to be limited to the examples that are now described; rather, the invention should be construed to include any and all applications provided herein and all equivalent variations within the skill of the ordinary artisan.

### Hypoxia

The Pasteur effect, which describes the increased conversion of glucose to lactate in hypoxic cells, has been considered a critical cellular metabolic adaptation to hypoxia for over a century. Increased glycolytic flux requires transcriptional activation of genes encoding glucose transporters and glycolytic enzymes. Hypoxia-inducible factor 1 (HIF-1)^{1,2} regulated the transcription of these downstream genes. As described in more detail-below, the gene encoding pyruvate dehydrogenase kinase 1 (PDK1) was identified as a direct target of HIF-1. PDK1 phosphorylates and inactivates pyruvate dehydrogenase (PDH), the enzyme that converts pyruvate to acetyl-coenzyme A, thereby inhibiting glucose metabolism via the tricarboxylic acid (TCA) cycle³. Under hypoxic conditions, HIF-1α-null mouse embryo fibroblasts undergo apoptosis that is associated with a dramatic increase in the level of reactive oxygen species (ROS). Forced expression of PDK1 prevents hypoxia-induced ROS generation and apoptosis and increases ATP levels. Without wishing to be bound to any particular theory, it is likely that a failure in the electron transport chain under hypoxic conditions necessitates the shunting of glucose metabolites away from the mitochondria by HIF-1-mediated PDK1 expression. This expression likely prevents the production of ROS and promotes ATP production through glycolysis.

### EXAMPLE 1

### PDK1 is Highly Induced by Hypoxia and is Responsive to MYC

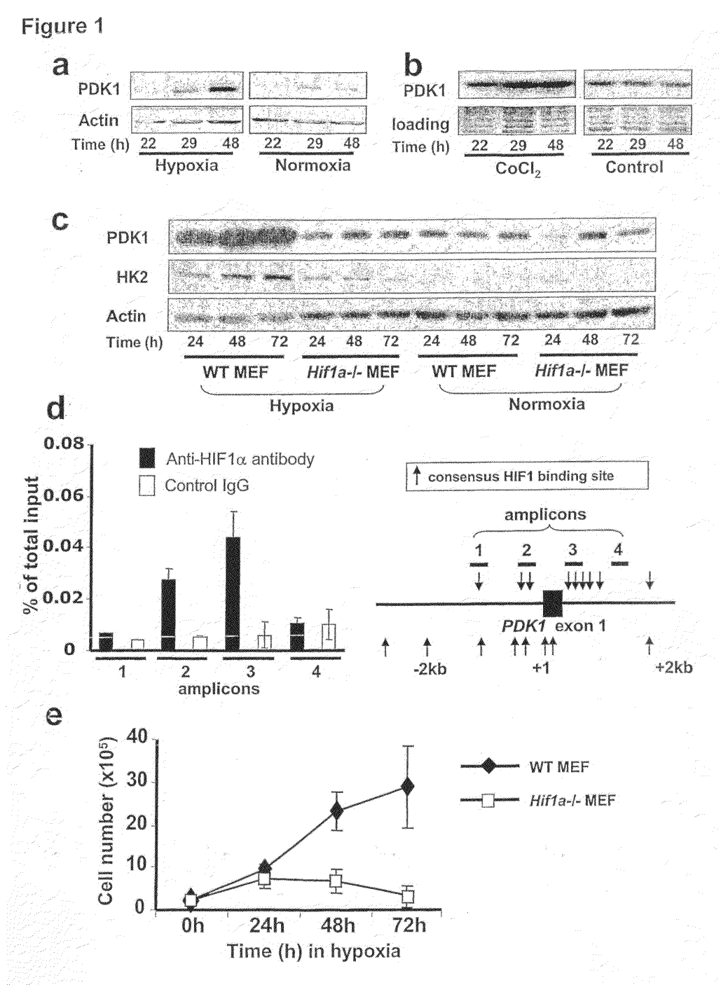

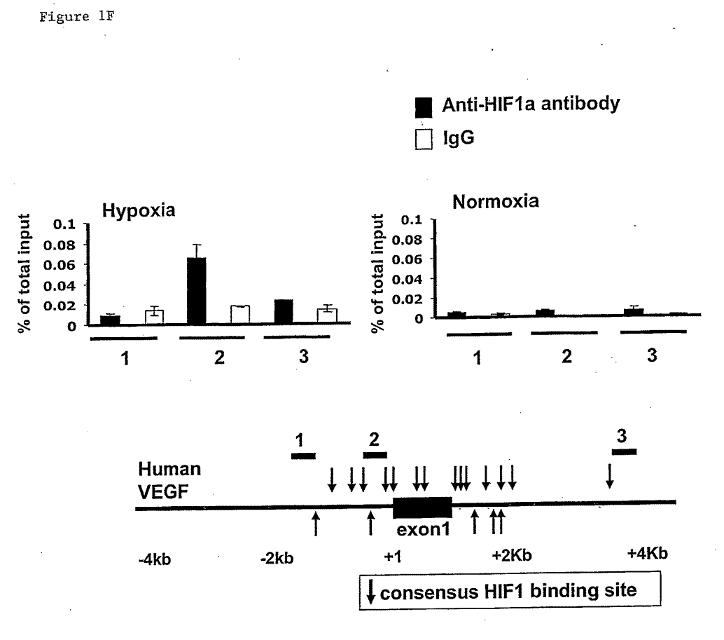

Microarray analysis was used to characterize gene expression in the human B lymphocyte cell line, P493-6, which contains a tetracycline-repressible MYC allele^{4,5}. This analysis identified genes responsive to both MYC and hypoxia PDK1 was identified as one gene that is highly induced by hypoxia. PDK1 was previously shown to be a potential MYC target⁶. Because of PDK1's involvement in the regulation of glucose metabolism by the TCA cycle, it was selected for further analysis. Hypoxic induction of PDK1 protein expression was demonstrated by immunoblot assay (FIG. 1A).

HIF-1 is a heterodimeric transcription factor, consisting of HIF-1α and HIF-1α subunits, which functions as a master regulator of oxygen homeostasis in all metazoan species^{7,8}. PDK1 levels were also increased in P493-6 cells exposed to CoCl₂ (FIG. 1B), which induces HIF-1 activity by inhibiting O₂-dependent degradation of the HIF-1α a subunit^{9,10}. To determine whether HIF-1 is necessary for PDK1 induction, HIF-1α-null (Hif1a^{-/-}) mouse embryo fibroblasts^{2,11} were analyzed. The dramatic increase in PDK1 levels in isogenic wild-type mouse embryo fibroblasts exposed to hypoxia did not occur in Hif1a^{-/-} mouse embryo fibroblasts (FIG. 1C). Similar results were obtained by immunoblot assay of hexokinase 2 (HK2), which is the product of a known HIF-1 target gene². To determine whether PDK1 is a direct target of HIF-1, chromatin immunoprecipitation (ChIP) was performed with an anti-HIF1 antibody, as described for anti-Myc antibody, using hypoxic P493-6 cells. The binding of HIF1 to a known HIF1 target, VEGF, was mapped. VEGF was bound by HIF1 in hypoxic chromatic but not in normoxic chromatin (FIG. 1F, FIG. 1D). In hypoxia, HIF-1a bound PDK1 in regions enriched with consensus HIF1 binding sites flanking exon1 (FIG. 1F). Taken together, these results demonstrate that PDK1 is a direct HIF-1 target gene.

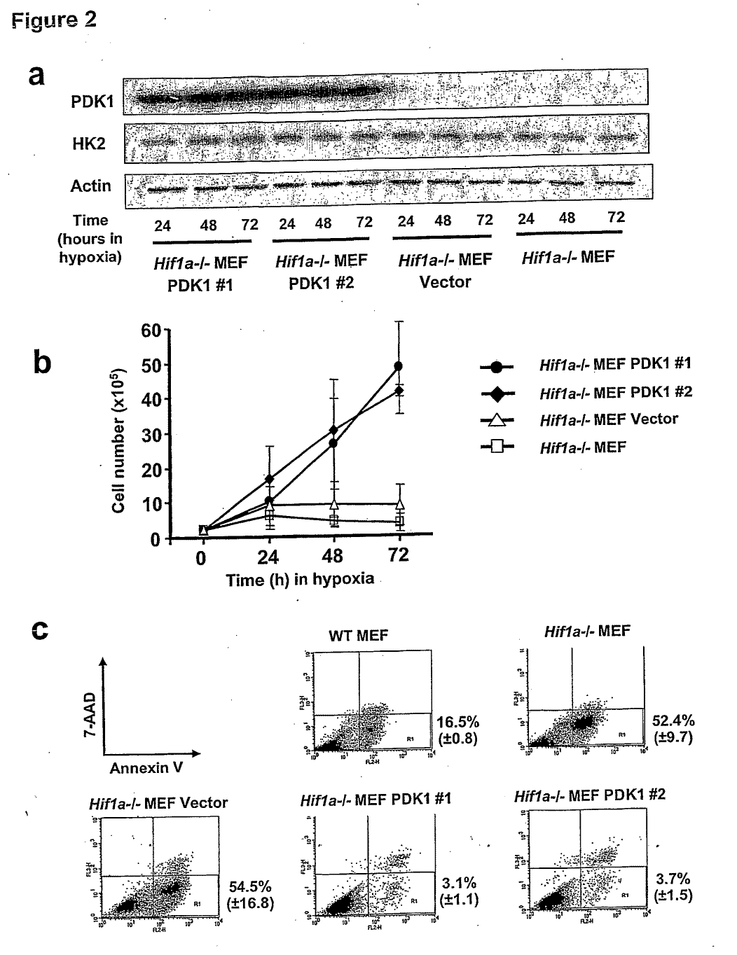

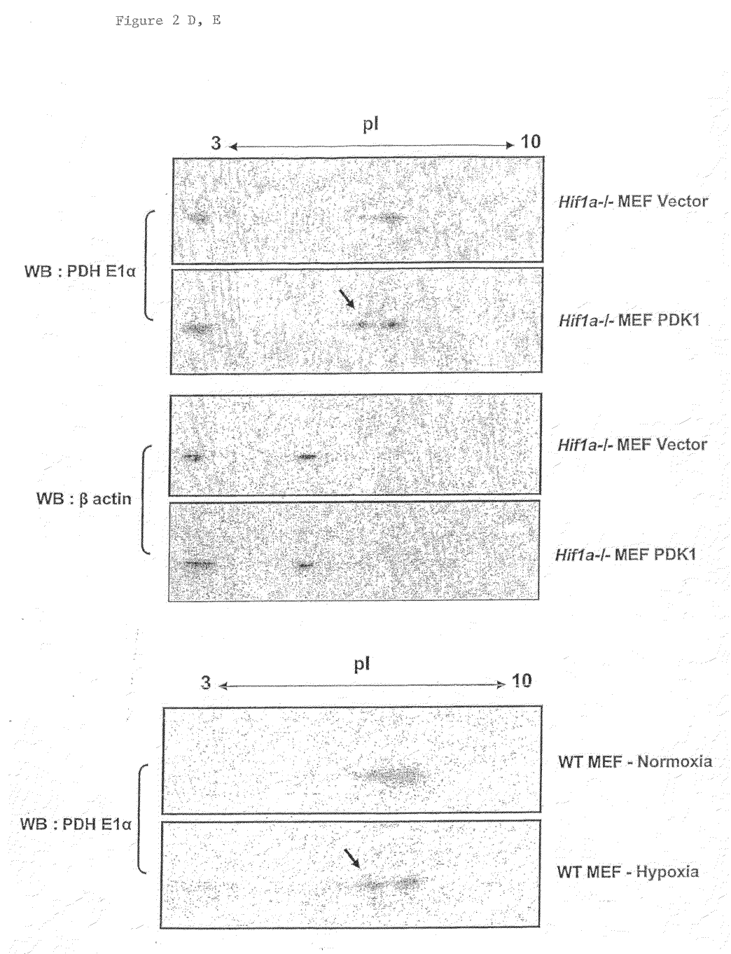

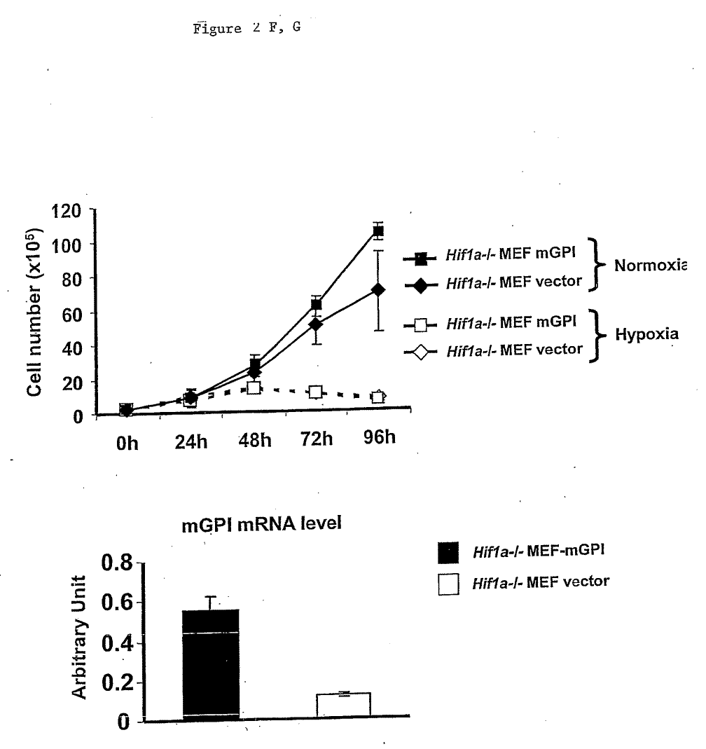

The proliferation of Hif1a^{-/-} embryonic stem cells maybe impaired when cultured under hypoxic conditions for 24-48 h^{2,12}. The proliferation of Hif1a^{-/-} mouse embryo fibroblasts were also impaired after forty-eight hours of hypoxia (FIG. 1E). A more striking defect was observed after seventy-two hours, with a reduction in cell number indicating cell death, which was confirmed by demonstration of a dramatic increase in apoptosis (FIG. 2C). In contrast, immortalized wild-type mouse embryo fibroblasts are able to proliferate in hypoxia, presumably because T antigen inactivates the RB-mediated G1 checkpoint elicited in moderately hypoxic cells¹³.

### EXAMPLE 2

### PDK1 Inhibits the Hypoxic Cell Death of HIF-1α-null MEFs

To determine whether active suppression of the TCA cycle and stimulation of glycolysis via inactivation of PDH by PDK1 is required for cell survival under hypoxic conditions, Hif1a^{-/-} cell pools with forced overexpression of PDK1 by independent retroviral infections were generated (FIG. 2A). This overexpression resulted in increased PDH E1α subunit phosphorylation, which was also observed in hypoxic wild-type mouse embryo fibroblasts (FIGS. 2D and 2E). Intriguingly, forced PDK1 expression was sufficient to permit the proliferation of hypoxic Hif1a^{-/-} mouse embryo fibroblasts (FIG. 2B) and to protect them from hypoxia-induced apoptosis (FIG. 2C). In contrast, forced expression of the murine glycolytic enzyme glucose phosphate isomerase (mGPI) could not rescue hypoxic Hif1a^{-/-} mouse embryo fibroblasts (FIGS. 2F and 2G).

### EXAMPLE 3

### HIF-1-Induced PDK1 Activity Reduces ROS Production

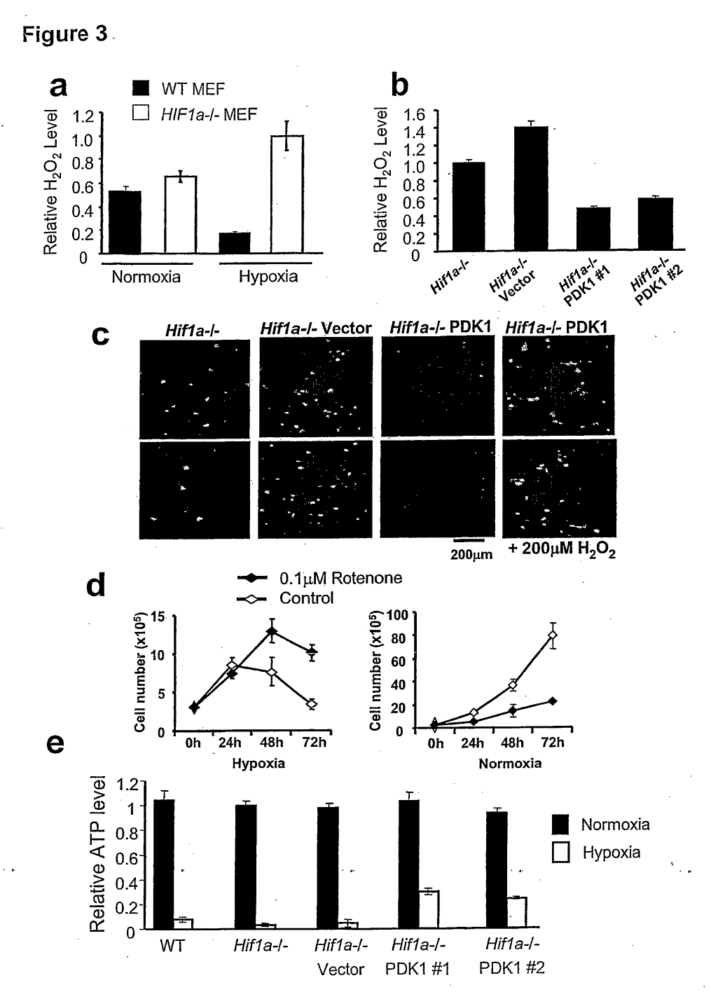

The observation that PDK1 rescued hypoxic HIF-1α-null mouse embryo fibroblasts suggested that PDK1-mediated inactivation of the PDH complex; that PDK1 shunted pyruvate away from the TCA cycle toward glycolysis; and that these activities were sufficient for the survival of hypoxic cells. Limited O₂ availability may lead to increased ROS production due to ineffective electron transfer in the mitochondria if flux through the TCA cycle is not attenuated^{14,15}. Increased ROS levels would, in turn, trigger apoptosis¹⁴. As shown in FIG. 3A, hypoxia caused an increase in intracellular H₂O₂ in Hif1a^{-/-} mouse embryo fibroblasts in sharp contrast to the reduction in H₂O₂ levels that was observed when wild type mouse embryo fibroblasts were exposed to hypoxia. These data, taken together with the demonstration that forced PDK1 expression prevented hypoxia-induced apoptosis of Hif1a^{-/-} mouse embryo fibroblasts suggested that HIF-1-induced PDK1 activity reduces ROS production. As shown in FIG. 3B, production of H₂O₂ in hypoxic Hif1a^{-/-} mouse embryo fibroblasts was significantly decreased by forced PDK1 expression. To further confirm that PDK1 reduces ROS production, intracellular oxidants were examined by staining cells with H₂DCFDA, which is oxidized by ROS to the highly fluorescent DCF. DCF fluorescence was markedly diminished by forced PDK1 expression in Hif1a^{-/-} mouse embryo fibroblasts (FIG. 3C). Without wishing to be bound by theory, it is likely that inhibition of mitochondrial electron transport also rescues the Hif1a^{-/-} MEFs. While myxothiazol and antimycin A were toxic to both normoxic and hypoxic Hif1a^{-/-} MEFs. Rotenone was able to rescue hypoxic Hif1a^{-/-} mouse embryo fibroblasts, whereas rotenone inhibited proliferation of normoxic Hif1a^{-/-} MEFs (FIG. 3D). These results support a novel regulatory mechanism for hypoxic adaptation in which PDK1 inactivates the PDH complex and inhibits the TCA cycle, thereby attenuating reactive oxygen species production and perhaps increasing glycolysis and ATP production by shunting pyruvate toward lactate production (FIG. 7).

### EXAMPLE 4

### Reduction of PDH E1 a Expression by siRNA Rescued Hif1a^{-/-} MEFs

ATP production in Hif1a^{-/-} mouse embryo fibroblasts was significantly reduced in hypoxia as compared with wild-type mouse embryo fibroblasts (FIG. 3E). In contrast, hypoxic Hif1a^{-/-} mouse embryo fibroblasts with forced PDK1 expression had an elevated ATP level as compared with hypoxic wild-type mouse embryo fibroblasts (MEFs) (FIG. 3E). Forced PDK1 expression caused a greater production of lactate by Hif1a^{-/-} mouse embryo fibroblasts even under normoxic conditions, under which PDH was found to have increased phosphorylation and was presumably inactivated (FIG. 2E). Further corroborating the role of PDH as a relevant target of PDK1 in hypoxia, reduction of PDH E1 a expression by small interference RNA partially rescued Hif1a^{-/-} MEFs at twenty-four and forty-eight hours as compared with control siRNAs. It is notable however that Hif1a^{-/-} MEFs treated with targeted or: control siRNAs died 72 hours after electroporation. These observations suggest that forced PDK1 expression rescued Hif1a^{-/-} MEFs by inactivating PDH, decreasing ROS production and increasing ATP production.

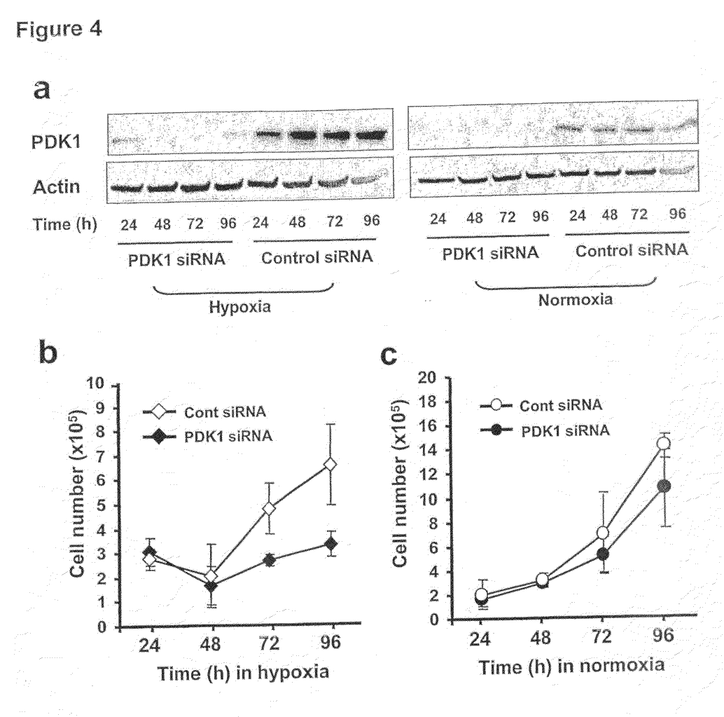

To determine whether PDK1 is necessary for hypoxic adaptation of P493-6 cells, which express predominantly PDK1 (as compared to one of the other three PDK isoforms), the expression of PDK1 was reduced by RNA interference (FIG. 4A). The growth of P493-6 cells in hypoxia was impaired by small interfering RNA (siRNA) directed against PDK1 as compared to cells treated with a scrambled control siRNA that did not reduce PDK1 expression (FIG. 4B). These results are consistent with the hypothesis that PDK1 is necessary for the proliferation of P493-6 cells under hypoxic conditions.

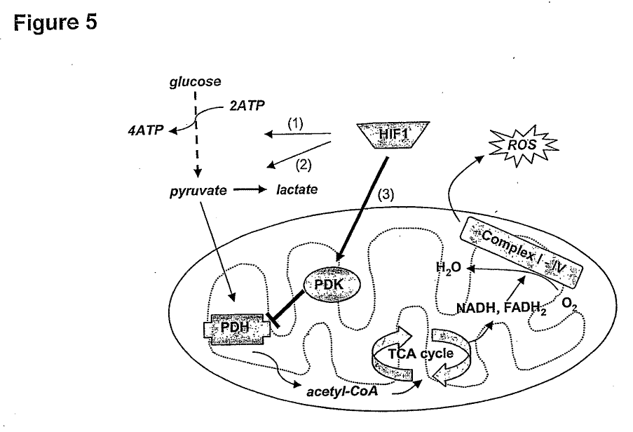

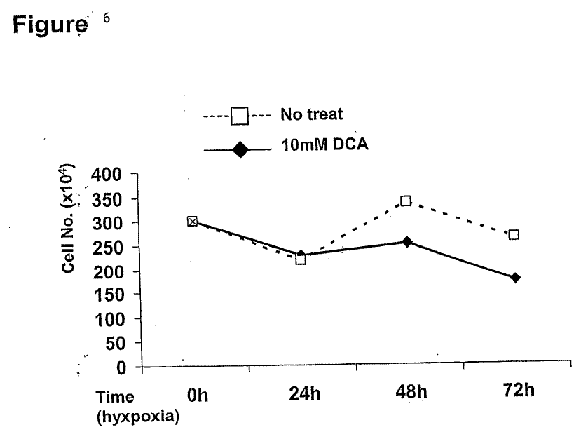

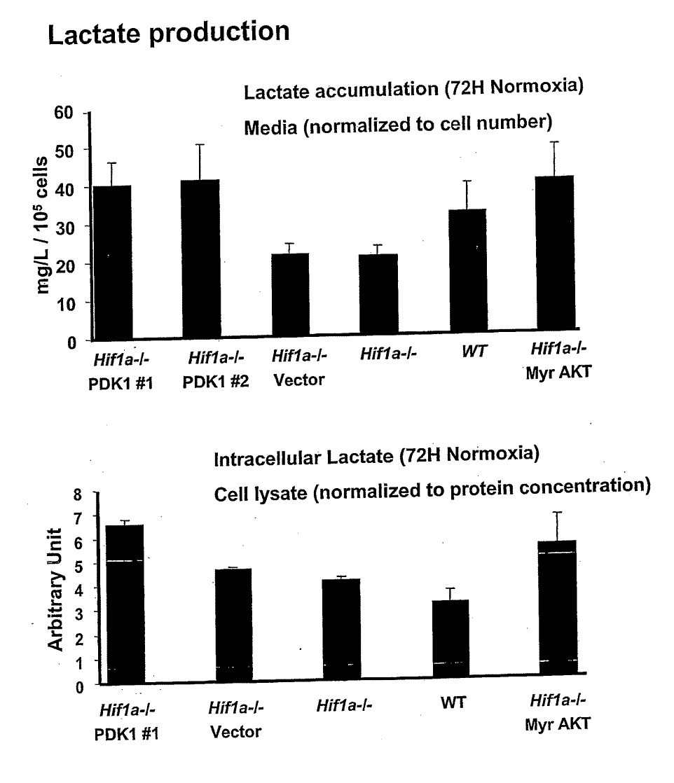

The finding that PDK1 was sufficient to rescue hypoxic cells that lack the expression of HIF-1α or HIF-2α supports a novel regulatory mechanism for hypoxic adaptation. While the possibility that PDK1 may have phosphorylation targets other than PDH that promote survival in hypoxia cannot be ruled out, the results reported herein strongly suggest that suppression of the TCA cycle and of reactive oxygen species production and stimulation of ATP production by HIF-1-mediated induction of PDK1 is crucial for the survival of hypoxic cells. Thus, HIF-1 plays three critical roles in the metabolic switch from oxidative to glycolytic metabolism by inducing expression of: (i) PDK1 to block the conversion of pyruvate to acetyl CoA; (ii) lactate dehydrogenase A to convert pyruvate to lactate; and (iii) upstream glucose transporters and glycolytic enzymes to increase flux from glucose to pyruvate (FIG. 5). It is likely that the induction of PDK1 is necessary to prevent excessive and potentially lethal mitochondrial reactive oxygen species production as well as shunting pyruvate toward glycolysis for ATP production under hypoxia (FIG. 5). These results indicate that therapeutic approaches that induce apoptosis in hypoxic cancer cells by PDK inhibition are likely to be useful for the treatment of hypoxia-resistant neoplasias. Furthermore, it is likely that PDH inhibition will protect ischemic tissues from oxidative stress.

### EXAMPLE 5

### Dichloroacetate Inhibited the Survival of Neoplastic Cells in Hypoxia

To determine whether a compound that inhibits pyruvate dehydrogenase kinase would reduce survival in neoplastic cells under hypoxic conditions, P493-6 cells were cultured under hypoxic conditions in the presence or the absence of dichloroacetate. Dichloroacetate is currently the most effective treatment for congenital lactic acidosis (CLA). People affected by CLA have defective PDC enzymes, which are required for efficient cellular respiration. As shown in FIG. 6, dichloroacetate inhibited the survival of P493-6 cells under hypoxic conditions.

The experiments described above were carried out using the following materials and methods.

### Cell Culture and Hypoxic Exposures

Wild type and Hif1a^{-/-} MEFs were immortalized by SV-40 large T antigen and maintained DMEM (GIBCO/BRL) with 15% fetal bovine serum (FBS) (GIBCO/BRL), 1 mM sodium pyruvate (Sigma, St. Louis, Mo.), non-essential amino acids (Sigma, St. Louis, Mo.) and 1% penicillin-streptomycin (GIBCO/BRL)¹¹. The human Burkitt's lymphoma cell line P493-6 was generated and maintained as described^{4,5}. Non-hypoxic cells were maintained at 37° C. in a 5% CO₂ incubator. Hypoxic cells were maintained in a control atmosphere chamber (Plas-Labs) at 37° C. Oxygen tension was monitored by a calibrated Series 200 Percent oxygen analyzer (Alpha Omega Instruments).

### Vectors and Retrovirus Infection

Verified full-length cDNA clones for human PDK1 (GenBank Accession No. NM_{—}002610) were purchased from Open biosystems. Full-length human PDK1 cDNA was cloned into a retroviral vector, pMSCVpuro (Clontech, Palo Alto, Calif.) (pMSCVpuro-PDK1). Retroviruses were produced by transfecting the pMSCVpuro-PDK1 or empty pMSCVpuro vector into the ecotropic Phoenix packaging cell line. Hif1a^{-/-} MEFs were infected with retroviruses in the presence of an anti-heparin agent, 8 μg/ml POLYBRENE (Sigma, St. Louis, Mo.). Infected cells were selected with 21 μg/ml puromycin (Sigma, St. Louis, Mo.).

### Western Blot Analysis

Proteins extracted from MEFs or P493-6 cells were loaded and resolved on 10% SDS-PAGE gel. Polyclonal anti-PDK1 antibody (Stressgen Bioreagents, Victoria, BC), polyclonal anti-HK2 antibody (Santa Cruz Biotechnology Inc, Santa Cruz, Calif.) and monoclonal anti-beta actin antibody (Sigma, St. Louis, Mo.) were used for immunoblotting.

### Cell Proliferation and Apoptosis

For the cell proliferation assay, 2×10⁵ MEFs were plated in 10 cm dish 1 day before hypoxic exposure (0.5% O₂). At indicated times, cells were trypsinized and viable cells were counted. Apoptotic rate was measured by Annexin V-PE Apoptosis Detection kit (BD Biosciences, Mountain View, Calif.)) according to the manufacturer's instructions.

### Reactive Oxygen Species Measurement

Intracellular hydrogen peroxide level was measured using a resorufin production assay, the AMPLEX RED Hydrogen Peroxide Assay kit (Molecular Probes, Eugene, Oreg.) according to the manufacturer's instructions. Briefly, total cell lysates were harvested at seventy-two hours after hypoxic incubation was initiated inside a hypoxic chamber and the reactions were initiated immediately by adding AMPLEX RED reaction mixture. Fluorescence was measured using a fluorescence plate reader, a CYTOFLUOR 2300 (Millipore. Billerica, Mass.). Fluorescence levels were normalized to the protein concentration.

Intracellular reactive oxygen species production was also measured by staining with dichlorodihydrofluorescein diacetate (H₂DCFDA, Molecular Probe, Eugene, Oreg.). After seventy-two hours of hypoxic incubation, cells were loaded with 5 μM H₂DCFDA for one hour, washed in PBS and incubated with fresh media without H₂DCFDA for 30 minutes. DCF fluorescence was visualized using an inverted fluorescence microscope, the Axiovert 200 (Zeiss, Oberkochen, Germany).

siRNA Experiments

siRNA targeting human PDK1 was designed and purchased from Dharmacon Research Inc (Lafayette, Colorado). 3×10⁶ P493-6 cells were electroporated (1500 uF and 240 volts) with 100 nM of PDK1 siRNA (5′-CUACAUGAGUCGCAUUUCAdTdT-3′) or scrambled control siRNA (5′-CACGCUCGGUCAAAAGGUUdTdT-3′) in a 4 mm cuvette (BTX) using a Gene Pulser Xcell (Bio-Rad, Hercules, Calif.). The following day, 5×10⁵ viable cells were subjected to hypoxic exposure (0.1% O₂). At indicated times, viable cells were counted for growth curve, and the cellular proteins were harvested for Western blot analysis.

### Two-Dimensional Electrophoresis

After washes with low salt wash buffer, the cells were extracted in lysis buffer (8 M urea, 4% CHAPS, 1.5% 3-10 IPG buffer, protease and phosphatase inhibitor cocktail). The crude cell homogenate was sonicated on ice and the first-dimension isoelectric focusing and second dimension electrophoresis were performed as described with modifications. After second dimension electrophoresis, proteins were transferred to nitrocellulose membrane and immunoblotted with monoclonal anti-PDH E1α antibody (Molecular Probes) or monoclonal anti-β-actin antibody (Sigma).

### Microarray Analysis

mRNA was isolated from P493-6 cells and subjected to microarray analysis Affymetrix oligonucleotide microarray analysis by using an HG_U133A chip as described¹⁷.

### Murine Glucose Phosphate Isomerase Experiments

The retroviral vector encoding murine GPI (pHygroMarX II-mGPI) and the control pHygroMarXII vector were kindly provided by H. Kondoh (Cancer Research UK, London Research Institute). Retroviruses were produced by transfecting the pHygroMarX II-mGPI or empty vector into the ecotropic Phoenix packaging cell line. Hif1α−/− MEFs were infected with retroviruses in the presence of 8 μg/ml polybrene (Sigma). Infected cells were selected with 400 μg/ml hygromycin (Sigma). The real-time RT-PCR was performed using TaqMan one-step RT-PCR master mix kit (PE Applied Biosystems) with probes and primers as described 5. The expression level of 18S RNA was used for normalization. All PCR reactions were performed in duplicate.

### REFERENCES

1. Seagroves, T. N. et al., Mol Cell Biol 21, 3436-44 (2001).

2. Lyer, N. V. et al., Genes Dev 12, 149-62 (1998).

3..Holness, M. J. et al., Biochem Soc Trans 31, 1143-51 (2003).

4. Schubmacher, M. et al., Curr Biol 9, 1255-8 (1999).

5. Kim, J. W. et al., Mol Cell Biol 24, 5923-36 (2004).

6. Li, Z. et al., Proc Natl Acad Sci USA 100, 8164-9 (2003).

7. Semenza, G. L., Physiology (Bethesda) 19, 176-82 (2004).

8. Schofield, C. J. et al., Nat Rev Mol Cell Biol 5, 343-54 (2004).

9. Wang, G. L. et al., Proc Natl Acad Sci USA 90, 4304-8 (1993).

10. Maxwell, P. H. et al., Nature 399, 271-5 (1999).

11. Feldser, D. et al., Cancer Res 59, 3915-8 (1999).

12. Carmeliet, P. et al., Nature 394, 485-90 (1998).

13. Gardner, L. B. et al., J Biol Chem 276, 7919-26 (2001).

14. Balaban, R. S. et al., Cell 120, 483-95 (2005).

15. Yankovskaya, V. et al., Science 299, 700-4 (2003).

16. Dang, C. V. et al., Trends Biochem Sci 24, 68-72 (1999).

17. Chou, W. C. et al., Proc Natl Acad Sci USA 101, 4578-83 (2004).

1. A method of treating or preventing a neoplasia in a subject, the method comprising administering to a subject in need of such treatment an effective amount of a pharmaceutical composition comprising a PDK inhibitor in a pharmaceutically acceptable inhibitor.

2. The method of claim 1, wherein the PDK inhibitor is a small molecule.

3. The method of claim 1, wherein the PDK inhibitor is selected from the group consisting of dichloroacetate, 2,2-dichloroacetophenone, and (+)-1-N-[2,5-(S, R)-dimethyl-4-N-(4-cyanobenzoyl )piperazine]-(R)-3,3,3-trifluoro-2-hydroxy-2-methylpropanamide.

4. The method of claim 1, wherein the PDK inhibitor is an inhibitory nucleic acid molecule that reduces PDK1 expression.

5. The method of claim 4, wherein the inhibitory nucleic acid molecule is a small interfering RNA (siRNA), antisense RNA, or other nucleic acid inhibitor of PDK expression.

6. The method of claim 5, wherein the inhibitory nucleic acid molecule is an siRNA that inhibits PDK expression.

7. A method of treating or preventing a neoplasia, the method comprising administering to a patient in need of such treatment an effective amount of a pharmaceutical composition that decreases the expression of a PDK polypeptide, that decreases the biological activity of a PDK polypeptide, and/or that decreases the expression of a PDK nucleic acid molecule.

8-9. (canceled)

10. A method of treating or preventing a neoplasia in a subject, the method comprising administering to a subject in need of such treatment an effective amount of a pharmaceutical composition comprising a PDK inhibitory nucleic acid molecule formulated in a pharmaceutically acceptable carrier, and/or a pharmaceutical composition comprising a PDK1 inhibitor in a Pharmaceutically acceptable composition.

11-33. (canceled)

34. A vector comprising a nucleic acid molecule that encodes a PDK1 inhibitory nucleic acid molecule of claim 26.

35-41. (canceled)

42. A host cell comprising the vector of claim 38.

43-48. (canceled)

49. A pharmaceutical composition for the treatment of a neoplasia, the composition comprising a pharmaceutical excipient and an effective amount of a small compound that inhibits a PDK biological activity.

50-54. (canceled)

55. A PDK1 biomarker purified on a solid substrate.

56. A diagnostic kit for the diagnosis of a neoplasia in a subject comprising a PDK nucleic acid molecule, or fragment thereof, and written instructions for use of the kit for detection of a neoplasia.

57-62. (canceled)

63. A method of determining the severity of a neoplasia in a patient, the method comprising determining PDK1, PDK2, PDK3, or PDK4 activity or expression in a patient sample, wherein an increase in the level of PDK1, PDK2, PDK3, or PDK4 activity or expression relative to the level of activity or expression in a reference indicates the severity of neoplasia in the patient.

64-68. (canceled)

69. A method of identifying a candidate compound that ameliorates a neoplasia, the method comprising contacting a neoplastic cell that expresses a PDK polypeptide under hypoxic conditions with a candidate compound, and comparing the level of expression of the polypeptide in the cell contacted by the candidate compound with the level of polypeptide expression in a control cell not contacted by the candidate compound, wherein a decrease in the expression of the PDK polypeptide identifies the candidate compound as a candidate compound that ameliorates a neoplasia.

70-90. (canceled)

91. A method of enhancing cell survival in a subject in need thereof, the method comprising administering to a subject in need of such treatment an effective amount of a pharmaceutical composition comprising a PDH inhibitor in a pharmaceutically acceptable inhibitor.

92-97. (canceled)

98. A method of treating or preventing cell damage related to hypoxia in a subject, the method comprising administering to a subject in need of such treatment an effective amount of a pharmaceutical composition that decreases the expression of a PDH polypeptide, and/or a pharmaceutical composition that decreases the biological activity of a PDH polypeptide.

99-100. (canceled)

101. The method of claim 99, wherein the method comprises administering fluoropyruvate, bromopyruvate, or 2-oxo-3-butynoic acid.

102-109. (canceled)

110. A PDH nucleic acid inhibitor comprising at least ten nucleic acids complementary to a nucleic acid molecule encoding a PDH polypeptide, wherein the nucleic acid molecule reduces expression of the PDH polypeptide in a cell.

111-126. (canceled)

127. A method of identifying a candidate compound that enhances survival in a cell at risk of cell death related to hypoxia, the method comprising contacting a cell that expresses a PDH polypeptide under hypoxic conditions with a candidate compound, and comparing the level of expression of the polypeptide in the cell contacted by the candidate compound with the level of polypeptide expression in a control cell not contacted by the candidate compound, wherein a decrease in the expression of the PDH polypeptide identifies the candidate compound as a candidate compound that ameliorates a neoplasia.

128-136. (canceled)
